(19) United States
(12) Patent Application Publication
(10) Pub. No.: US 2022/0331266 A1
(43) Pub. Date: Oct. 20, 2022

(54) CBD COMPOSITION

(71) Applicant: CANNPAL ANIMAL THERAPEUTICS LIMITED, New South Wales (AU)

(72) Inventors: Layton MILLS, New South Wales (AU); Margaret Ann CURTIS, New South Wales (AU)

(21) Appl. No.: 17/621,330

(22) PCT Filed: Jun. 26, 2020
(86) PCT No.: PCT/AU2020/050666

§ 371 Date: Dec. 21, 2021

(30) Foreign Application Priority Data
Jun. 26, 2019 (AU) 2019902236

(51) Int. Cl. A61K 31/05; A61K 36/185; A61P 17/00; A61K 31/355; A61K 31/575; A61P 29/00

(52) U.S. Cl. CPC A61K 31/05; A61K 36/185; A61P 17/00; A61K 31/355; A61K 31/575; A61P 29/00; A61K 31/19

(57) ABSTRACT

The application provides a composition comprising cannadibiol, a fatty acid component comprising a balanced amount of omega-3 and omega-6 fatty acids, and a sterol and optionally a vitamin E compound. The composition has been shown to be well tolerated and supportive when administered to subjects suffering from or at risk of developing a skin disorder and/or an inflammatory condition, such as atopic dermatitis. Also disclosed are methods of supporting the immune system and skin health of a subject involving administration of the compositions.

This application claims priority to Australian provisional patent application no. 2019902236 (filed on 26 Jun. 2019), the entire contents of which are incorporated herein by reference.

### FIELD OF INVENTION

The invention relates to a composition comprising cannabidiol (CBD). The invention also relates to methods for its use in treating subjects (including companion animals) in need of skin or immune support, such as those suffering from or at risk of developing a skin disorder and/or an inflammatory condition.

### BACKGROUND

Hemp, or industrial hemp, is a variety of the Cannabis Sativa plant species which has been used for thousands of years for its reported benefits for overall health and wellbeing due to the plant's unique phytochemical compositions. One of the most widely used phytochemicals from the hemp plant is CBD.

CBD is reported to have a variety of biological activities due to its activity on the endocannabinoid system, in particular activity at the cannabinoid type 2 receptor (CB2). The endocannabinoid system is highly conserved and there is growing and continuing interest in understanding the potential of endocannabinoid system modulation to achieve various health benefits. Unlike some other cannabinoids, CBD modulates the endocannabinoid system without any reported significant psychoactive effects.

Other phytochemicals found in the hemp plant such as flavonoids, terpenes, vitamins, phytosterols and certain fatty acids have also been reported for their therapeutic benefits, however due to traditional cultivation practises including outdoor cultivation where temperature and weather patterns can fluctuate, it's difficult to produce a composition containing CBD together with other phytochemicals from the hemp plant with consistency and repeatability in the levels required to provide a consistently effective combination of active ingredients.

Due to recent and evolving regulatory changes, there is growing interest in using Hemp extracts and/or CBD as a pharmaceutical, veterinary and nutraceutical agent to treat a range of conditions or support functional health.

There is a continuing need to develop CBD-based treatment options, such as a composition that may assist in treating skin disorders and/or inflammation. Further, there is also a need to provide standardised compositions comprising compounds reported in Hemp extracts and, which can be reliably produced with a consistent active ingredient profile.

### SUMMARY

In one aspect, the invention provides a composition comprising:
at least about 0.1% by weight cannabidiol (CBD);
at least about 40% by weight of a fatty acid component comprising a balanced ratio of an omega-3 fatty acid and an omega-6 fatty acid; and
a sterol.

In some embodiments, the composition may further comprise a vitamin E compound.

In some embodiments, the composition may comprise:
at least about 0.1% by weight CBD;
at least about 40% by weight of a fatty acid component comprising a balanced ratio of an omega-3 fatty acid and an omega-6 fatty acid;
β-sitosterol
β-caryophyllene
a vitamin E compound; and
an antioxidant.

In another aspect, the invention provides a method for treating a subject with a skin disorder and/or inflammatory condition, comprising administering an effective amount of the composition of the invention to a subject in need thereof.

In a further aspect, the invention provides use of one or more of CBD, an omega-3 fatty acid, an omega-6 fatty acid and/or a sterol in the preparation of a medicament for treating a subject with a skin disorder and/or an inflammatory condition, wherein the medicament comprises at least about 0.1% by weight of CBD, at least about 40% by weight of a fatty acid component comprising an omega-3 fatty acid and an omega-6 fatty acid, and a sterol.

Before describing the present invention in detail, it is to be understood that this invention is not limited to particularly exemplified embodiments, such as the compositions, methods, uses and processes, which may, of course, vary. It is also to be understood that the terminology used herein is for the purpose of describing particular embodiments of the invention only and is not intended to be limiting.

Any embodiment described herein shall be read independently or in combination with any other embodiment unless specifically stated otherwise.

All publications, patents and patent applications that may be cited herein, whether supra or infra, are hereby incorporated by reference in their entirety.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

The term “administering” refers to providing the composition to a subject suffering from or at risk of the disorders(s) and/or condition(s) to be treated.

By “effective amount” it is meant an amount sufficient that, when administered to the subject, an amount of the composition is provided to achieve an effect. In the case of a therapeutic method, this effect may be the treatment of a skin disorder and/or inflammation. Therefore, the “effective amount” may be a “therapeutically effective amount”. By “therapeutically effective amount” it is meant an amount sufficient that when administered to a subject an amount of composition is provided to treat the disorder or a symptom of the disorder.

As used herein, the terms “treating”, “treatment”, “treat” and the like mean affecting a subject (e.g. a patient), tissue or cell to obtain a desired pharmacological and/or physiological effect. The effect may be prophylactic in terms of completely or partially preventing, or reducing the severity of, a disease or associated symptom, and/or may be therapeutic in terms of a partial or complete cure of a disease. For example, a reference to “treating” inflammation therefore encompasses: (a) arresting the progress of the disease, e.g. preventing worsening of a symptom or complication over time; (b) relieving or ameliorating the effects of inflammation, i.e. causing an improvement of at least one symptom or complication of inflammation; (c) preventing additional symptoms or complications of inflammation from developing; and/or (d) preventing inflammation or a symptom associated with inflammation from occurring in a subject. In addition, a reference to “treating” a skin disorder therefore encompasses: (a) arresting the progression of a skin disorder either in severity or in terms of area affected; (b) relieving or ameliorating the severity of one or more symptoms of the skin disorder; (c) preventing additional symptoms associated with the skin disorder from developing; and/or (d) preventing or slowing the occurrence or re-occurrence of the skin disorder in the subject.

It must be noted that as used herein and in the appended claims, the singular forms “a,” “an,” and “the” include plural reference unless the context clearly dictates otherwise. Thus, for example, a reference to “a fatty acid” and/or “at least one fatty acid” may include one or more fatty acids, and so forth.

Unless defined otherwise, all technical and scientific terms used herein have the same meanings as commonly understood by one of ordinary skill in the art to which this invention belongs. Although any materials and methods similar or equivalent to those described herein can be used to practice or test the present invention, the preferred materials and methods are now described.

The term “(s)” following a noun contemplates the singular or plural form, or both.

The term “and/or” can mean “and” or “or”.

Unless the context requires otherwise, all percentages referred to herein are percentages by weight of the composition.

Unless the context requires otherwise, all amounts referred to herein are intended to be amounts by weight.

Various features of the invention are described with reference to a certain value, or range of values. These values are intended to relate to the results of the various appropriate measurement techniques, and therefore should be interpreted as including a margin of error inherent in any particular measurement technique. Some of the values referred to herein are denoted by the term “about” to at least in part account for this variability. The term “about”, when used to describe a value, may mean an amount within ±25%, ±10%, ±5%, ±1% or ±0.1% of that value.

The term “comprising” is used in an inclusive sense, i.e. to specify the presence of the stated features but not to preclude the presence or addition of further features in various embodiments of the invention. When interpreting statements in this specification that include that term, the features, prefaced by that term in each statement, all need to be present but other features can also be present. Related terms such as “comprise” and “comprised” are to be interpreted in the same manner.

The term “pharmaceutically acceptable” in the context of a form of a compound or an additive to the composition, is intended to mean that the form of the compound or the additive to the composition is suitable for use in a pharmaceutical sense. Therefore, pharmaceutically acceptable forms and/or additives are non-toxic to the subject in the amounts in which they are present in the compositions described herein. In some embodiments, the composition of the invention is a nutraceutical composition. It will be appreciated that any ingredient that is pharmaceutically acceptable will also be suitable for nutraceutical use.

The term “veterinary acceptable” in the context of a form of a compound or an additive to the composition, is intended to mean that the form of the compound or the additive to the composition is suitable for use in a veterinary sense. Therefore, veterinary acceptable forms and/or additives are non-toxic to the non-human subject in the amounts in which they are present in the composition described herein.

The term “nutraceutically acceptable” in the context of a form of a compound or an additive to the composition, is intended to mean that the form of the compound of the additive to the composition is suitable for use in a nutraceutical sense. Therefore, nutraceutically acceptable forms and/or additives are non-toxic to the subject in the amounts in which they are present in the composition described herein. It will be appreciated that all pharmaceutically acceptable forms and additives will typically also be nutraceutically acceptable.

The term “cannabinoid” as used herein relates to any compound that has activity involving the endocannabinoid system and that has been reported in an extract of a Cannabis plant, whether derived from a Cannabis plant or synthetically created.

The term “phytocannabinoid” refers to cannabinoids derived from a Cannabis plant.

The term “cannabinomimetic” refers to a compound that has activity involving the endocannabinoid system other than an endocannabinoid or phytocannabinoid.

The term “cannabinoid fraction” is used to describe the combination of cannabinoids present in the Cannabis extract.

### BRIEF DESCRIPTION OF THE DRAWINGS

Embodiments of the invention will be further described with reference to the following non-limiting drawings, in which:

FIG. 1 shows a chart of the CADESI-4 score change in 13 dogs included in the study described in Example 2. The data are provided as a comparison of pretreatment CADESI-4 score to the score 56 days after treatment with placebo or a composition of the invention (DC_ISO and DC_WHE).

FIG. 2 shows a chart of the change in monocyte chemoattractant protein-1 (MCP-1) also known as chemokine (C-C motif) ligand 2 (CCL2) in CAD dogs treated with either placebo or a composition of the invention in the study of Example 2.8 over the 56 day treatment period.

FIG. 3 shows a chart of the change in keratinocyte-derived chemokine (KC) also known as CXCL1 in CAD dogs treated with either placebo or a composition of the invention in the study of Example 2.8 over the 56 day treatment period.

FIG. 4 shows a chart of the change in interleukin-8 (IL-8) in CAD dogs treated with either placebo or a composition of the invention in the study of Example 2.8 over the 56 day treatment period.

FIG. 5 shows a chart of the change in chemokine (C-X-C motif) ligand 1 (CXCL1) in CAD dogs treated with either placebo or a composition of the invention in the study of Example 2.9 over the 56 day treatment period.

FIG. 6 shows a chart of the change in hepatocyte growth factor (HGF, Hepatopoietin-A) in CAD dogs treated with either placebo or a composition of the invention in the study of Example 2.9 over the 56 day treatment period.

FIG. 7 shows a chart of the change in receptor for advanced glycation end products (RAGE) in CAD dogs treated with either placebo or a composition of the invention in the study of Example 2.9 over the 56 day treatment period.

### DESCRIPTION OF EMBODIMENT(S)

The invention provides a composition comprising:
at least about 0.1% by weight CBD;
at least about 40% by weight of a fatty acid component comprising a balanced ratio of an omega-3 fatty acid and an omega-6 fatty acid; and
a sterol.

The combination of ≥0.1 wt % CBD, ≥40 wt % of a fatty acid component comprising a balanced ratio of an omega-3 fatty acid and an omega-6 fatty acid, and a sterol may provide an alternative formulation useful in the treatment of subjects suffering from or at risk of developing a skin disorder and/or an inflammatory condition; or to support health skin and immune function. The composition may be used alone or as part of an adjuctive therapy. The composition may be a pharmaceutical composition, a veterinary composition and/or a nutraceutical composition.

The composition comprises standardised concentrations of each component and may be prepared by combination of ingredients derived from various natural sources. However, it will be appreciated that the composition comprises the various compounds in amounts that are not present in any one natural source. Rather, for at least canine subjects, it is shown that the combination of compounds present in the composition provides a formulation that is well tolerated and provides a biologically meaningful effect.

Cannabidiol (CBD)

CBD has the following structure:

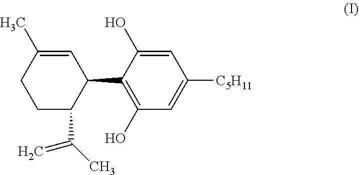

The composition may comprise CBD in any amount from at least about 0.1% to less than about 60%. In some embodiments, the minimum amount of CBD may be at least about 0.1%, 0.15%, 0.2%, 0.25%, 0.3%, 0.35%, 0.4%, 0.45% or 0.5%. In some embodiments, the maximum amount of CBD may be not more than about 10%, 9%, 8%, 7%, 6%, 5%, 4%, 3%, 2%, 1%, 0.9%, 0.8%, 0.7% or 0.6%. The composition may comprise CBD in an amount from any of these minimum amounts to any of these maximum amounts, for example, from about 0.1% to about 10% or about 0.2% to about 1%.

The CBD may be provided from a natural or a synthetic source, or a combination sources, including semi-synthetic sources. CBD provided from natural sources may be isolated, and then combined. Alternatively, extracts (enriched in CBD) may be used to provide the CBD. Synthetic or semi-synthetic or isolated natural CBD may be added to a Cannabis extract to enrich the extract in CBD. These mixtures may be prepared by any means known in the art.

CBD derived from a natural source may be present as a mixture with cannabidiolic acid (CBDA). CBDA is a natural carboxylated form of CBD and decarboxylates under many quantification techniques. Accordingly, any concentration of CBD described here when derived from a natural source includes the combined concentration of CBD and CBDA. Preferably, the composition comprises a majority of CBD relative to any amount of CBDA present.

In some embodiments, the CBD is provided in a purity of greater than about 98%, 98.5%, 99%, 99.5% or 99.9%. When obtained from a natural source, the purified CBD may comprise a combination of CBD and CBDA in any of these purities. The purified CBD may comprise minor amounts of other cannabinoids, such as up to about 0.5%, 0.4%, 0.3%, 0.2% or 0.1%. Other cannabinoids that may be present may be selected from Δ⁹-tetrahydrocannabinol (THC), Δ⁸-tetrahydrocannabinol (Δ8-THC), cannabidivirin (CBDV) and CBD-C4. In some embodiments, the total amount of THC and Δ8-THC is not more than about 0.1% (typically not more than 0.03%), and/or the amount of CBDV is not more than about 0.5% (typically not more than 0.4%), and/or the total amount of CBD-C4 is not more than about 0.5% (typically not more than 0.2%). Other cannabinoids (i.e. other than THC, Δ8-THC, CBDV and CBD-C4 may be present in an amount of not more than 0.1%, typically less than 0.05%.

In some embodiments, the CBD is provided in the form of a Cannabis extract, preferably a Hemp extract.

To date, over 100 cannabinoids have been identified in Cannabis extracts. A list of these cannabinoids may be found in Mahmoud A. El Sohly and Waseem Gul, “Constituents of Cannabis Sativa” In Handbook of Cannabis Roger Pertwee (Ed.) Oxford University Press (2014) (ISBN: 9780199662685). Cannabinoids that have been identified in Cannabis plants include: Cannabigerol (E)-CBG-C5, Cannabigerol monomethyl ether (E)-CBGM-C5 A, Cannabigerolic acid A (Z)-CBGA-C5 A, Cannabigerovarin (E)-CBGV-C3, Cannabigerolic acid A (E)-CBGA-C5 A, Cannabigerolic acid A monomethyl ether (E)CBGAM-C5 A and Cannabigerovarinic acid A (E)-CBGVA-C3 A; (±)-Cannabichromene CBC-C5, (±)-Cannabichromenic acid A CBCA-C5 A, (±)-Cannabivarichromene, (±)-Cannabichromevarin CBCV-C3, (±)-Cannabichromevarinic acid A CBCVA-C3 A; (−)-Cannabidiol CBD-C5, Cannabidiol momomethyl ether CBDMC, Cannabidiol-C4 CBD-C4, (−)-Cannabidivarin CBDV-C3, Cannabidiorcol CBD-CI, Cannabidiolic acid CBDA-C5, Cannabidivarinic acid CBDVA-C3; Cannabinodiol CBND-C5, Cannabinodivarin CBND-C3; Δ9-Tetrahydrocannabinol Δ9-THC-C5, Δ9-Tetrahydrocannabinol-C4 Δ9-THC-C4, Δ9-Tetrahydrocannabivarin Δ9-THCV-C3, Δ9-Tetrahydrocannabiorcol Δ9-THCO-CI, Δ9-Tetrahydrocannabinolic acid A Δ9-THCA-C5 A, Δ9-Tetrahydrocannabinolic acid B Δ9-THCA-C5 B, Δ9-Tetrahydrocannabinolic acid-C4 A and/or B Δ9-THCA-C4 A and/or B, Δ9-Tetrahydro-cannabivarinic acid A Δ9-THCVA-C3 A, Δ9-Tetrahydrocannabiorcolic acid A and/or B Δ9-THCOA-CI A and/or B), (−)-Δ8-trans-(6aR,10aR)-Δ8-Tetrahydrocannabinol Δ8-THC-C5, (−)-Δ8-trans-(6aR,10aR)-Tetrahydrocannabinolic acid A Δ8-THCA-C5 A, (−)-(6aS,10aR)-Δ9-Tetrahydrocannabinol (−)-cis-Δ9-THC-C5; Cannabinol CBN-C5, Cannabinol-C4 CBN-C4, Cannabivarin CBN-C3, Cannabinol C2 CBN-C2, Cannabiorcol CBN-CI, Cannabinolic acid A CBNA-C5 A, Cannabinol methyl ether CBNM-C5, (−)-(9R,10R)-trans-Cannabitriol (−)-trans-CBT-C5, (+)-(9S,10S)-Cannabitriol (+)-trans-CBT-C5, (±)-(9R,10S/9S,10R)); Cannabitriol (±)-cis-CBT-C5, (−)-(9R,10R)-trans-10-O-Ethyl-cannabitriol (−)-trans-CBT-OEt-C5, (±)-(9R,10R/9S,10S)-Cannabitriol-C3 (±)-trans-CBT-C3, 8,9-Dihydroxy-Δ6a(10a)-tetrahydrocannabinol 8,9-Di-OH-CBT-C5, Cannabidiolic acid A cannabitriol ester CBDA-C5 9-OH-CBT-C5 ester, (−)-(6aR,9S,10S,10aR)-9,10-Dihydroxyhexahydrocannabinol, Cannabiripsol, Cannabiripsol-C5, (−)-6a,7,10a-Trihydroxy-Δ9-tetrahydrocannabinol (−)-Cannabitetrol, 10-Oxo-Δ6a(10a)tetrahydrocannabinol (OTHC); (5aS,6S,9R,9aR)-Cannabielsoin CBE-C5, (5aS,6S,9R,9aR)-C3-Cannabielsoin CBE-C3, (5aS,6S,9R,9aR)-Cannabielsoic acid A CBEA-C5 A, (5aS,6S,9R,9aR)-Cannabielsoic acid B CBEA-C5 B; (5aS,6S,9R,9aR)-C3-Cannabielsoic acid B CBEA-C3 B, Cannabiglendol-C3 OH-iso-HHCV-C3, Dehydrocannabifuran DCBF-C5, Cannabifuran CBF-C5, (−)-Δ7-trans-(1R,3R,6R)-Isotetrahydrocannabinol, (±)-Δ7-1,2-cis-(1R,3R,6S/1S,3S,6R)-Isotetrahydrocannabivarin, (−)-Δ7-trans-(1R,3R,6R)-Isotetrahydrocannabivarin; (±)-(1aS,3aR,8bR,8cR)-Cannabicyclol CBL-C5, (±)-(1aS,3aR,8bR,8cR)-Cannabicyclolic acid A CBLA-C5 A, (±)-(paS,3aR,8bR,8cR)-Cannabicyclovarin CBLV-C3; Cannabicitran CBT-C5; Cannabichromanone CBCN-C5, CannabichromanoneC3 CBCN-C3, and Cannabicoumaronone CBCON-C5.

In addition to a cannabinoid fraction, Cannabis extracts (including Hemp extracts) may also comprise a diverse array of secondary metabolites, including terpenes and terpenoids, sterols, triglycerides, alkanes, squalenes, tocopherols, carotenoids and alkaloids. The mix of these secondary metabolites varies depending on several factors, including Cannabis variety, part of the Cannabis plant extracted, method of extraction, processing of the extract, and season.

There are several varieties of Cannabis plant, which have been described under two distinct naming conventions. One of these conventions identifies three distinct species of Cannabis plant, namely Cannabis sativa linnaeus, Cannabis indica LAM., and Cannabis ruderalis. Another convention identifies all Cannabis plants as belonging to the Cannabis sativa L. species, with the various varieties divided amongst several subspecies, including: Cannabis sativa ssp. sativa and ssp. indica. As used herein, the term “Cannabis” refers to any and all of these plant varieties. In preferred embodiments of the invention, the Cannabis extract is an extract of a Hemp plant (Hemp extract).

The Cannabis extract may be prepared by any means known in the art. The extracts may be formed from any part of the Cannabis plant. Extracts may be formed by contacting an extractant with a leaf, seed, trichome, flower, keif, shake, bud, stem or a combination thereof. Any suitable extractant known in the art may be used, including, for example, alcohols (e.g. methanol, ethanol, propanol, butanol, propylene glycol, etc.), water, hydrocarbons (e.g. butane, hexane, etc.), oils (e.g. olive oil, vegetable oil, essential oil, etc.), a polar organic solvent (e.g. ethyl acetate, polyethylene glycol, etc.) or a supercritical fluid (e.g. liquid CO₂). The extractant may be completely or partially removed prior to incorporation of the Cannabis extract into the composition, or it may be included in the composition to also a CB2 agonist, an omega-3 fatty acid, an omega-6 fatty acid and an antioxidant act as a carrier. The extractant may be removed by heating the extract optionally under reduced pressure (e.g. under vacuum). It will be appreciated that some of the more volatile plant metabolites (such as terpenes) may also be removed with the extractant. Accordingly, in some embodiments, removing the extractant may enrich the cannabinoid fraction of the extract. In some embodiments, the extract is filtered to remove particulate material, for example, by passing the extract through filter paper or a fine sieve (e.g. a sieve with pore sizes of 5 μm).

In some embodiments, the Cannabis extract is formed by applying heat and pressure to the plant material. Typically, in these embodiments, no extractant is required.

In some embodiments, the Cannabis extract is a Cannabis oil. As used herein, a “Cannabis oil” is an extract formed by contacting at least a part of a Cannabis plant with an oil. The extracting oil may optionally be removed. Extracting oils may be selected from olive oil, hemp oil, sesame oil, coconut oil, vegetable oil, canola oil, grape seed oil, almond oil, medium-chain triglyceride (MCT) oil, and any other edible oil, or a combination thereof.

The cannabinoid fraction typically accounts for the majority of the compounds present in the Cannabis extract.

In some embodiments, the Cannabis extract may comprise about 35% to about 95% by weight cannabinoids, for example, about 40% to about 90%, about 45% to about 70% or about 45% to about 55% by weight of the Cannabis extract. In some embodiments, the Cannabis extract comprises about 5% to about 65% by weight of non-cannabinoids, for example, about 5% to about 50%, about 10% to about 40% by weight or about 15% to about 30% by weight non-cannabinoids.

In some embodiments, the cannabinoid fraction comprises CBD as the main cannabinoid. The Cannabis fraction may comprise CBD in a minimum amount of at least about 40%, 45%, 50%, 55% or 60%. The Cannabis fraction may comprise a maximum amount of CBD of not more than about 97%, 96%, 95%, 94%, 93%, 92%, 91%, 90%, 85% or 80%. The cannabinoid fraction may comprise CBD from any of these minimum amounts to any of these maximum amounts, for example, about 40 to about 97% or about 50 to about 90%.

Typically, the Cannabis extract may also comprise other cannabinoids in addition to CBD. These cannabinoids include Δ⁹-tetrahydrocannabinol (THC), Δ⁸-tetrahydrocannabinol (d8-THC), Δ⁹-tetrahydrocannabinolic acid (THCA), Δ⁹-tetrahydrocannabivarin (THCV), (−)-cannabidivarin (CBDV), cannabinodiol (CBN) and cannabigerol (CBG). Each of these cannabinoids may be present in an amount from 0.001% to 40% by weight of the Cannabis extract. For example, THC may be present in the cannabinoid fraction in an amount of not more than about 20%, 15%, 10%, 5% or 1%. CBN may be present in the cannabinoid fraction in an amount of not more than 0.5%, 0.4%, 0.3%, 0.2% or 0.1%. CBN may be present in the cannabinoid fraction in an amount of 0.001 to 0.5% or 0.001 to 0.1% by weight. CBG may be present in the cannabinoid fraction in an amount of at least 0.3% by weight of the extract, for example, 0.3 to 10% or 0.35 to 5% by weight of the extract.

In some embodiments, certain cannabinoids may be absent from the Cannabis extract, or present in non-detectable amounts (e.g. less than 0.001% by weight of the analyte). For example, in some embodiments, the composition is free of THC, such as less than about 1% or 0.1% of THC.

Fatty Acid Component

The fatty acid component of the composition comprises a balanced ratio of an omega-3 fatty acid and an omega-6 fatty acid.

An omega-3 fatty acid is an unsaturated fatty acid with a double carbon-carbon bond 3 atoms in from its terminal carbon atom. An omega-6 fatty acid is an unsaturated fatty acid with a double carbon-carbon bond 6 atoms in from its terminal carbon atom.

Omega-3 and omega-6 fatty acids are important in animal metabolism. In particular, α-linolenic acid (ALA; 18:3n-3) and linoleic acid (LA; 18:2n6) are essential fatty acids for a range of mammalian species, including dogs and humans. ALA is a building block of more complex omega-3 fatty acids, including stearidonic acid (18:4n-3), eicosatetraenoic acid (20:4n-3), eicopentaenoic acid (EPA; 20:5n-3) and docosahexanenoic acid (DHA; 22:6n-3). LA contributes to other important omega-6 fatty acids, including γ-linolenic acid (GLA; 18:3n6), dihomo-γ-linolenic acid (DGLA; 20:3n-6) and arachidonic acid (AA; 20:4n6).

These omega-3 and omega-6 fatty acids in turn are biosynthetic precursors to several classes of important metabolites, including eicosanoids, endocannabinoids and lipoxins.

Omega-3 and omega-6 fatty acids are also involved in the biosynthetic pathways for various pro-inflammatory and anti-inflammatory biomarkers and signalling compounds, such as prostaglandins, leukotrienes, thromboxanes and resolvins. For example, linoleic acid (omega-6) is a biosynthetic precursor to various pro-inflammatory compounds, such as prostaglandin 2 (PG2), leukotriene B4 (LTB4) and thromboxane A (TXA), while α-linolenic acid (omega-3) is a biosynthetic precursor to various anti-inflammatory compounds, such as prostigalandin 3 (PG3), leukotriene B5 (LTB5), thromboxane A3 (TXA3), resoMn E1 (RvE1), resoMn E2 (RvE2), resoMn D3 (RvD3) and resoMn D4 (RvD4).

The fatty acid component of the composition may therefore also assist managing inflammation in a subject following administration. Reducing inflammation may assist support the immune system of the subject. In addition, it has been reported that dietary omega-3 and omega-6 fatty acids may act as homeostatic regulators of endocannabinoids, in part since they are endocannabinoid biosynthetic precursors. It is therefore believed that the combination of the fatty acid composition and CBD in the composition assist in modulating the endocannabinoid system of the subject.

Any fatty acid described herein may be included in the composition in a free acid form or in the form of an ester, such as a triglyceride. It will therefore be understood that a reference to a “fatty acid”-including references to omega-3 fatty acids and omega-6 fatty acids—includes the free fatty acid and an ester thereof. Fatty acid esters include glyceryl esters, such as monoglycerides, diglycerides and triglycerides. The fatty acid residues included in a diglyceride and/or triglyceride may be the same or different and may be selected from any of the fatty acids described herein.

It is believed that the relative proportion of omega-3 fatty acid to omega-6 fatty acid may be important for fatty acid metabolism, CBD bio-absorption and health outcomes achieved for the subject.

It is desirable that the composition includes the fatty acid component as (i) omega-3 fatty acids and omega-6 fatty acids are associated with a range of health benefits, and (ii) the efficacy of some active pharmaceutical, veterinary and/or nutraceutical ingredients is enhanced by co-administration with a fatty acid, for example as the fatty acid may enhance bioavailability of the active ingredient(s).

The composition comprises the omega-3 and omega-6 fatty acids in a balanced ratio. In some embodiments, the minimum ratio of omega-3 to omega-6 fatty acids may be at least about 0.8:1, 0.9:1, 0.95:1 or 1:1 based on the weight of the fatty acid component. The maximum ratio of omega-3 to omega-6 fatty acids may be up to about 1.2:1, 1.15:1, 1.1:1, 1.05:1 or 1:1 based on the weight of the fatty acid component. The ratio of omega-3 fatty acids to omega-6 fatty acids may be from any of these minimum ratios to any of these maximum ratios, for example, from about 0.8:1 to about 1.2:1, about 0.9:1 to about 1.1:1 or about 1:1 to about 1.15:1.

The fatty acid component comprises an omega-3 fatty acid. The minimum omega-3 fatty acid content of the fatty acid component may be at least about 15%, 20%, 25% or 30% based on the weight of the fatty acid component. The maximum omega-3 fatty acid content of the fatty acid component may be up to about 65%, 60%, 55%, 50% or 45% based on the weight of the fatty acid component. The omega-3 fatty acid content of the fatty acid component may be from any of these minimum amounts to any of these maximum amounts, for example, from about 15% to about 65% or from about 25% to about 50%.

In some embodiments, the composition comprises an omega-3 fatty acid selected from ALA, EPA, stearidonic acid and DHA and combinations thereof.

In some embodiments, the omega-3 fatty acid comprises ALA in a major amount. Reference to “major amount” refers to the component (eg ALA) of the fraction (eg omega-3 fatty acids) present in the highest concentration. The minimum concentration of ALA may be at least about 80%, 90%, 95% or 97% based on the weight of omega-3 fatty acids. The maximum concentration of ALA may be up to about 100%, 99.9%, 99.5%, 99%, or 98% based on the weight of omega-3 fatty acids. The concentration of ALA based on the weight of omega-3 fatty acids may be from any of these minimum concentrations to any of these maximum concentrations, for example, from about 97% to about 100%.

The fatty acid component comprises an omega-6 fatty acid. The minimum omega-6 fatty acid content of the fatty acid component may be at least about 15%, 20% or 25% based on the weight of the fatty acid component. The maximum omega-6 fatty acid content of the fatty acid component may be up to about 65%, 60%, 55%, 50% or 45% based on the weight of the fatty acid component. The omega-6 fatty acid content of the fatty acid component may be from any of these minimum amounts to any of these maximum amounts, for example, from about 15% to about 65% or from about 25% to about 50%.

In some embodiments, the composition comprises an omega-6 fatty acid selected from LA, GLA, DGLA and AA and combinations thereof.

In some embodiments, the omega-6 fatty acid comprises LA in a major amount. Reference to “major amount” refers to the component (eg LA) of the fraction (eg omega-6 fatty acids) present in the highest concentration. The minimum concentration of LA may be at least about 80%, 90% or 94% based on the weight of omega-6 fatty acids. The maximum concentration of LA may be up to about 100%, 99% or 98% based on the weight of omega-6 fatty acids. The concentration of LA based on the weight of omega-6 fatty acids may be from any of these minimum concentrations to any of these maximum concentrations, for example, from about 90% to about 98%.

The ratio of omega-3 to omega-6 fatty acids is typically determined based on the weight of each fatty acid of each class in the fatty acid component. However, in some embodiments, the ratio of omega-3 and omega-6 fatty acids may be determined based on the ratio of the major component of each type. Accordingly, in some embodiments, the ratio of ALA and LA is balanced. The ratio of ALA to LA may be any of the balanced ratios of omega-3 to omega-6 fatty acids described herein. In some embodiments, the balanced ratio of ALA to LA is about 1:1 on a weight for weight basis, for example, from 0.9:1 to 1.1:1.

In some embodiments, the fatty acid component comprises GLA. The fatty acid component may comprise γ-linolenic acid in an amount from about 1% to about 5%.

In some embodiments, the fatty acid component may comprise stearidonic acid. The fatty acid component may comprise stearidonic acid in an amount of 0-2.5%.

In some embodiments, the fatty acid component comprises ALA. The fatty acid component may comprise α-linolenic acid in an amount of at least about 20% and/or up to about 50%, for example from about 20% to about 50% or about 30% to about 48%.

In some embodiments, the fatty acid component comprises LA. The fatty acid component may comprise LA in an amount of at least about 25% and/or in an amount of up to 43%, for example, from about 25% to about 43% or about 28.5% to about 42.5%.

In some embodiments, the fatty acid component comprises palmitic acid, stearic acid, oleic acid, LA, ALA, GLA and stearidonic acid. The fatty acid component may comprise these fatty acids in any of the following amounts:

about 3-9% palmitic acid,

about 1-6% stearic acid,

about 8-24% oleic acid,

about 28-43% linoleic acid,

about 30-50% α-linolenic acid,

about 1-5% γ-linolenic acid and

0-2.5% stearidonic acid.

The composition comprises at least about 40% of the fatty acid component. In some embodiments, the composition comprises a minimum concentration of fatty acid component of at least about 40%, 45%, 50%, 55%, 60%, 65%, 70%, 75%, 80%, 85%, 90%, 91%, 92%, 93%, 94%, 95% or 96%. The composition may comprise a maximum concentration of the fatty acid component of up to about 99.9%, 99.5%, 99%, 98.5, 98%, 97.5%, 97% or 96.5%. The concentration of fatty acid component in the composition may be from any of these minimum values to any of these maximum values, for example from about 40% to about 99.99% or about 75% to about 98%.

In addition to the omega-3 and omega-6 fatty acids, the fatty acid component may comprise one or more additional fatty acids. The additional fatty acids may be saturated or unsaturated fatty acids.

Unsaturated fatty acids may comprise from 1 to n/2 double carbon-carbon bonds, wherein n is the number of carbon atoms in the fatty acid side chain. Typically, unsaturated fatty acids comprise from 1 to 10 double carbon-carbon bonds. The double carbon-carbon bonds may be cis or trans. Typically, the double carbon-carbon bonds are cis.

The additional fatty acid may be:
a short chain fatty acid (SCFA) comprising from 2 to 6 carbon atoms (inclusive of the carboxyl carbon);
a medium-chain fatty acid (MCFA) comprising from 7 to 13 carbon atoms (inclusive of the carboxyl carbon);
a long-chain fatty acid (LCFA) comprising from 14 to 22 carbon atoms (inclusive of the carboxyl carbon); and/or
a very long chain fatty acid (VLCFA) comprising 23 or more carbon atoms (inclusive of the carboxyl carbon), for example from 23 to 100 carbon atoms.

In some embodiments, the fatty acid component comprises a MCFA, a LCFA or a combination thereof.

Triglycerides comprising at least 1 MCFA may be referred to herein as a medium chain triglyceride (MCT). In some embodiments, the fatty acid component comprises a MCT.

In some embodiments, the fatty acid component comprises a fatty acid selected from one or more of the group consisting of butyric acid (4:0); caproic acid (6:0); caprylic acid (8:0); capric acid (10:0); undecanoic acid (11:0); lauric acid (12:0); tridecanoic acid (13:0); myristic acid (14:0); myristoleic acid (14:1); pentadecanoic acid (15:0); cis-10-pentadecanoic acid; cis-10-pentadecenoic acid; palmitic acid (16:0); palmitoleic acid (16:1n-9); hexadecenoic acid (16:1); hexadecadienoic acid (16:2); margic/heptadecanoic acid (17:0); cis-10-heptadecanoic acid; cis-10-heptadecenoic acid; margaroleic acid (17:1); stearic acid (18:0); vaccenic acid (18:1); oleic acid (18:1); elaidic acid (18:1); linoleic acid (LA; 18:2); linolelaidic acid (18:2n-6); linolenic acid (18:3) including α-linolenic acid (ALA) and γ-linolenic acid (GLA); octadecatrienoic acid (18:3); stearidonic acid (SDA; 18:4n-3); arachidic acid (20:0); eicosenoic acid (20:1) including gadoleic acid (20:1n-11), gondoic acid (20:1n-9) and paullinic acid (20:1n-7); eicosadienoic acid (20:1n-6); cis-11, 14, 17-eicosatrienoic acid; cis-8, 11, 14-eicosatrienoic acid; eicosatetraenoic acid; arachidic acid (AA; 20:0); eicosapentaenoic acid (20:5n-3); heneicosanoic acid (21:0); behenic acid (22:0); cetoleic/erucic acid (22:1n-9); dicosadienoic acid (22:2n-6); docosapentanoic/docosapentaenoic acid (DPA; 22:5); docosahexaenoic acid (DHA; 22:6n-3); tricosanoic acid (23:0); lignoceric acid (24:0); and nervonic acid (24:1n-9). These fatty acids may be present as free fatty acids or incorporated into a fatty acid ester.

The fatty acid component may comprise fatty acids derived from nature or produced synthetically (i.e. non-natural). In some embodiments, the composition comprises at least one fatty acid from a non-natural source.

In some embodiments, the fatty acid component comprises flax seed oil, hemp seed oil, fish oil, coconut oil, cocoa butter, palm kernel oil, palm oil, cottonseed oil, wheat germ oil, soybean oil, olive oil, corn oil, sunflower oil, safflower oil, canola oil, sesame oil, peanut oil, or a combination thereof. In some embodiments, the fatty acid component comprises a combination of 2, 3, 4 or more oils, such as any of the oils described herein. Typically, when the fatty acid component comprises a combination of oils these are mixed in a substantially equal proportions, for example in a ratio by volume of about 1:1 or 1:1:1 and so on.

Sterol

The composition comprises a sterol. The sterol may be a phytosterol, a zoosterol or a mycosterol. Typically, the sterol is a phytosterol. Phytosterols include β-sitosterol, campesterol and stigmasterol and derivatives thereof. In some embodiments, the composition comprises β-sitosterol.

The composition may comprise the sterol in a minimum amount of at least about 0.01%, about 0.05%, about 0.1%, about 0.2% or about 0.25%. The composition may comprise the sterol in a maximum amount of not more than about 15%, about 10%, about 5%, about 1%, about 0.5% or about 0.4%.

The composition may comprise the sterol in an amount from any of these minimum amounts to any of these maximum amounts, such as from about 0.01% to about 15% or about 0.2% to about 0.4%.

Other Ingredients

In addition to CBD, the fatty acid component and the sterol, the composition may comprise one or more further ingredients. The further ingredients may be selected from a Vitamin E compound, an antioxidant, a terpene, a food ingredient, a further pharmaceutical, veterinary or nutraceutical active ingredient, a carrier, a diluent and an excipient or a combination thereof.

Vitamin E compound(s)

Vitamin E comprises a mixture of tocopherols and tocotrienols. The tocopherols include α-tocopherol, β-tocopherol, γ-tocopherol and δ-tocopherol. The tocotrienols include α-tocotrienol, β-tocotrienol, γ-tocotrienol and δ-tocotrienol.

Advantageously, the vitamin E compound may provide several advantages to the composition, including supporting the skin health of the subject being administered the composition and may also assist in serving as an antioxidant both for the subject and to prevent fatty acid oxidation in the composition.

The vitamin E compound may be selected from any of the tocopherols, tocotrienols, a tocopherol derivative and a tocotrienol derivative, or a combination thereof. Tocopherol and tocotrienol derivatives include acetyl, glyceryl and phosphate derivatives.

In some embodiments, the vitamin E compound is α-tocopherol or a derivative thereof. In some embodiments, the vitamin E compound is a γ-tocopherol or a derivative thereof.

In some embodiments, the vitamin E compound is provided as vitamin E.

The vitamin E compound may be derived from a natural source or non-natural source. Non-natural sources of vitamin E compounds include synthetic and/or semi-synthetic tocopherols, tocotrienols, tocopherol derivatives and tocotrienol derivatives.

The composition may comprise the vitamin E compound in a minimum amount of at least about 0.1%, 0.5%, 1%, 2%, 3%, 4% or 5%. The composition may comprise the vitamin E compound in a maximum amount of not more than about 20%, 15%, 10%, 9%, 8%, 7% or 6%. The composition may comprise the vitamin E compound from any of these minimum amounts to any of these maximum amounts, for example, from about 0.1% to about 20% or about 1% to about 10%.

Antioxidant(s)

In some embodiments, the composition comprises an antioxidant to delay or prevent oxidation of the fatty acid component of the composition. Any compatible antioxidant may be included. The antioxidant may be selected from ascorbic acid, butylated hydroxyanisole, butylated hydroxytoluene (BHT), propyl gallate, a vitamin E compound, a mixture of tocopherois or a combination thereof. For example, the antioxidant may be Paramega™ which comprises a mixture of tocopherols and botanically sourced ingredients designed to stabilise omega-3 and omega-6 fatty acids. In some embodiments, the antioxidant is an extract of rosemary which comprises a mixture of tocopherols.

The composition may comprise the antioxidant in a stabilising amount. When the antioxidant is included in a stabilising amount it may be referred to as a stabiliser. A stabilising amount is any amount of the antioxidant effective to impede oxidation of the fatty acid component. The stabilising amount may therefore depend on the antioxidant or combination of antioxidants selected as well as other factors such as the fatty acids present and expected storage conditions for the composition.

In some embodiments, the minimum concentration of antioxidant may be at least about 0.0001%, 0.0005%, 0.001%, 0.005%, 0.01%, 0.05% or 0.1%. In some embodiments, the maximum concentration of antioxidant may be not more than about 20%, 15%, 10%, 9%, 8%, 7%, 6% or 5%. The composition may comprise the antioxidant in a concentration from any of these minimum concentrations to any of these maximum concentrations, for example, from about 0.0001% to about 20% or from about 0.05% to about 6%.

In some embodiments, the antioxidant comprises a vitamin E compound. In some embodiments, the antioxidant comprises an antioxidant other than a vitamin E compound.

Further Active Pharmaceutical, Veterinary and/or Nutraceutical Ingredient(s)

In some embodiments, the composition may comprise a further active pharmaceutical, veterinary and/or nutraceutical ingredient other than CBD, fatty acid component and the sterol. Any compatible active pharmaceutical, veterinary and/or nutraceutical ingredient may be included.

Typically, the further active pharmaceutical, veterinary and/or nutraceutical ingredient is fat soluble and/or fat dispersible. Fat dispersible active pharmaceutical, veterinary and/or nutraceutical ingredients may optionally comprise an emulsifier or solubility enhancer to assist form a dispersion with the fatty acid component.

Suitable examples include essential oils (e.g. frankincense oil), plant extracts (e.g. ginger root extract, turmeric root extract), terpenes (e.g. β-caryophyllene, α-pinene, β-caryophyllene oxide), flavonoids (e.g. quecertin, a cannflavin, etc.), bromelaine, essential amino acids including peptides comprising an essential amino acid, CB2 ligands (e.g. anandamide, 2-arachidonoylglycerol, 2 arachidonyl glyceryl ether, Δ⁹-tetrahydrocannabinol (THC), N-alkylamide, β-caryophyllene, 3,3′-diindolylmethane, AM-1221, AM-1235, AM-2232, UR-144, JWH-007, JWH-015, JWH-018, etc.), Janus Kinase (JAK) Inhibitors (e.g. JAK3 inhibitors), vitamins other than vitamin E, oclacitinib (eg Apoquel™, a corticosteroid (e.g. hydrocortisone, triamcinolone, methylprednisolone, prednisone, dexamethasone, etc.), an antihistamine (e.g. Diphenhydramine (Benadryl), Hydroxyzine (Atarax), Chlorpheniramine (Chlor-Trimeton), Loratadine (Claritin®), Cetirizine (Zyrtec®), etc.) a monoclonal antibody targeting specific chemokines or cytokines that contribute to a skin disorder and/or inflammation (e.g. Iokivetmab-Cytopoint®) and combinations thereof.

When present, the further active pharmaceutical, veterinary and/or nutraceutical ingredient is included in a therapeutically useful amount which is sufficient to provide a suitable dosage to a subject following administration. Accordingly, the composition may comprise an effective amount of a further active pharmaceutical, veterinary and/or nutraceutical ingredient.

In some embodiments, each further active pharmaceutical, veterinary and/or nutraceutical ingredient is present in a minimum amount of at least about 0.0001%, 0.0005%, 0.001%, 0.005%, 0.01% or 0.05%. In some embodiments, each further active pharmaceutical, veterinary and/or nutraceutical ingredient is present in a maximum amount of not more than about 10%, 5%, 1% or 0.5%. The composition may comprise each further active pharmaceutical, veterinary and/or nutraceutical ingredient from any of these minimum amounts to any of these maximum amounts, for example, from about 0.0001% to about 10% or from about 0.01% to about 1%.

In some embodiments, the further active pharmaceutical, veterinary and/or nutraceutical ingredient comprises a terpene. The terpene may comprise β-caryophyllene, α-pinene, β-caryophyllene oxide or a combination thereof. Compositions comprising β-caryophyllene may further comprise β-caryophyllene oxide. The composition may comprise the terpene in any amount described above for a further active pharmaceutical, veterinary and/or nutraceutical ingredient. Typically, the composition may comprise the terpene in an amount from about 0.001% to about 0.5% or from about 0.1% to about 0.4%.

In some embodiments, the further active pharmaceutical, veterinary and/or nutraceutical ingredient is β-caryophyllene. The composition may comprise β-caryophyllene in any amount described above for a further active pharmaceutical, veterinary and/or nutraceutical ingredient. Typically, the composition may comprise β-caryophyllene in an amount from about 0.001% to about 0.5% or from about 0.1% to about 0.4%.

In some embodiments, the further active pharmaceutical, veterinary and/or nutraceutical ingredient is α-pinene. The composition may comprise α-pinene in any amount described above for a further active pharmaceutical, veterinary and/or nutraceutical ingredient. Typically, the composition may comprise α-pinene in a maximum amount of up to about 1%, 0.5%, 0.25%, or 0.1%. The composition may comprise α-pinene in a minimum amount of at least about 0.001% or 0.01%. The composition may comprise α-pinene from any of these minimum amounts to any of these maximum amounts, for example from about 0.001% to about 1%.

In some embodiments, the composition is substantially free of α-pinene. In such embodiments, only trace levels of α-pinene may be included as introduced with a Cannabis plant extract or other component as an impurity, for example such compositions may comprise less than about 0.0001% α-pinene. In some embodiments, the composition comprises no added α-pinene. In some embodiments, the composition comprises no detectable level of α-pinene.

References to the various compounds described herein, such as CBD, fatty acids, the sterol and further active pharmaceutical, veterinary and/or nutraceutical ingredient(s), include the relevant compound and pharmaceutically, veterinary and/or nutraceutically acceptable salts, tautomers, solvates, polymorphs and/or stereoisomers thereof.

The various compounds may be provided as salts which are pharmaceutically, veterinary and/or nutraceutically acceptable. Examples of pharmaceutically and veterinary acceptable salts include salts of pharmaceutically, veterinary and/or nutraceutically acceptable cations such as sodium, potassium, lithium, calcium, magnesium, ammonium and alkylammonium; acid addition salts of pharmaceutically, veterinary and/or nutraceutically acceptable inorganic acids such as hydrochloric, orthophosphoric, sulfuric, phosphoric, nitric, carbonic, boric, sulfamic and hydrobromic acids; or salts of pharmaceutically, veterinary and/or nutraceutically acceptable organic acids such as acetic, propionic, butyric, tartaric, maleic, hydroxymaleic, fumaric, citric, lactic, mucic, gluconic, benzoic, succinic, oxalic, phenylacetic, methanesulfonic, trihalomethanesulfonic, toluenesulfonic, benzenesulfonic, isethionic, salicylic, sulphanilic, aspartic, glutamic, edetic, stearic, palmitic, oleic, lauric, pantothenic, tannic, ascorbic, valeric and orotic acids. Salts of amine groups (if present) may also comprise quaternary ammonium salts in which the amino nitrogen atom carries a suitable organic group such as an alkyl, alkenyl, alkynyl or aralkyl moiety.

The salts may be formed by conventional means, such as by reacting the free base form of the compound with one or more equivalents of the appropriate acid.

It should be understood that a reference to a pharmaceutically, veterinary and/or nutraceutically acceptable salt includes the solvent addition forms or crystal forms thereof, particularly solvates and/or polymorphs.

A “tautomer” is a structural isomer of a compound that is in equilibrium with another of the compound's structural isomers. This equilibrium is typically driven by thermodynamics making isolation of only one tautomer of a compound that exhibits tautomerism impossible by conventional techniques. To the extent that any of the present compounds exhibit tautomerism, it is intended that the invention includes all tautomers of the various compounds and derivatives thereof.

A “stereoisomer” is a spatial isomer of a compound which results in measurable changes to the rotation of polarised light passing through a solution of the compound. Stereoisomers include enantiomers, diastereomers, geometric isomers, rotamers including atropisomers and anomers.

The compound(s) may exist in unsolvated as well as solvated forms with acceptable solvents such as water, ethanol, and the like. Solvates contain either stoichiometric or non-stoichiometric amounts of a solvent. Hydrates are formed when the solvent is water. Alcoholates are formed when the solvent is an alcohol. In general, the solvated forms are considered equivalent to the unsolvated forms for the purposes of the compositions and methods provided herein.

The composition may typically be provided in the form or a liquid. However, in some embodiments, the composition may be provided in the form of a powder. Powdered forms of the composition may be achieved, for example, by encapsulation of CBD, the fatty acid component and the sterol along with any additional ingredient(s) within a matrix or shell comprising polysaccharides such as cyclodextrins, emulsifiers, proteins, peptides or combinations thereof. For example, encapsulation techniques are described in WO2001/074175. Alternatively, the composition may be encapsulated within liposomes.

The pharmaceutical, veterinary and/or nutraceutical compositions may be formulated, for example, by employing conventional solid or liquid vehicles or diluents, as well as pharmaceutical, veterinary and/or nutraceutical additives of a type appropriate to the mode of desired administration (for example, excipients, binders, preservatives, flavours, etc.) according to techniques such as those well known in the art of pharmaceutical formulation (see, for example, Remington: The Science and Practice of Pharmacy, 21st Ed., 2005, Lippincott Williams & Wilkins and/or Veterinary pharmacology and therapeutics, Riviere, J. (Ed.); Papich, Mark G., (Ed.); Wley-Blackwell; 2017). The additives may be any additive included in the United States Pharmacopeia/National Formulary (USP/NF), the British Pharmacopoeia (BP), the European Pharmacopoeia (EP), the Japanese Pharmacopoeia (JP) or the Chinese Pharmacopoeia (ChP). In some embodiments, the composition comprises an excipient which may be non-natural (e.g. synthetically produced).

The pharmaceutical compositions may be administered by any suitable route of administration, and may therefore be formulated in a form suitable for any such route of administration. For example, the route of administration may be oral, rectal, topical (including buccal and sub-lingual), vaginal or parenteral (including subcutaneous) administration. However, an advantage of the compositions of this invention, is the oral bioavailability of the active components.

The pharmaceutical, veterinary and/or nutraceutical compositions may be prepared in unit dosage form. In such form, the compositions are subdivided into unit doses containing appropriate quantities of the ingredient(s). The unit dosage form can be a packaged preparation, the package containing discrete quantities of preparation. The preparation may be a solid, such as packeted tablets, capsules (e.g. filled capsules), lozenges, powders in vials or ampoules, or a liquid, such as solutions, suspensions, emulsions, elixirs, tinctures or capsules filled with the same. Also, the unit dosage form can be a capsule, tablet, cachet, or lozenge itself, or it can be the appropriate number of any of these in packaged form.

For preparing pharmaceutical, veterinary and/or nutraceutical compositions described herein, pharmaceutically, veterinary and/or nutraceutically acceptable additives can be either solid or liquid. Solid form preparations include powders, tablets, pills, capsules, cachets, suppositories, lozenges and dispensable granules. A solid carrier can be one or more substances which may also act as diluents, flavouring agents, solubilisers, lubricants, suspending agents, binders, preservatives, tablet disintegrating agents, or an encapsulating material.

Suitable carriers include magnesium carbonate, magnesium stearate, talc, sugar, lactose, pectin, dextrin, starch (e.g. maize starch, potato starch, etc.), gelatin, tragacanth, methylcellulose, sodium carboxymethylcellulose, a low melting wax, cocoa butter, microcrystalline cellulose (MCC), silicified microcrystalline cellulose, powdered cellulose, alginate, polydextrose, calcium sulfate dihydrate, calcium hydrogen phosphate dihydrate, colloidal silicon dioxide, talc, hydroxypropyl methylcellulose (HPMC), hydroxypropyl methylcellulose acetate succinate (HPMCAS), polyvinylpyrrolidone (PVP), acrylates and methacrylates, polyethylene glycol (PEG), polyethylene oxide (PEO), acacia gum and the like. Tablets, powders, capsules, pills, cachets, and lozenges can be used as solid forms suitable for oral administration.

Liquid form preparations include oil solutions, dispersions, suspensions, and emulsions, for example, water-in-oil and oil-in-water emulsions. Liquid preparations are preferred for embodiments involving sub-lingual administration.

Liquid forms of the composition may be sterile. Sterile liquid form compositions include sterile solutions, suspensions, emulsions, syrups and elixirs. The ingredient(s) may be suspended in a pharmaceutically, veterinary and/or nutraceutically acceptable carrier, such as sterile water, sterile organic solvent or a mixture of both.

Aqueous solutions can be prepared by mixing the ingredient(s) in water with an emulsifier and adding suitable colorants, flavours, stabilising and thickening agents, as desired. Aqueous suspensions can be made by dispersing the finely divided active ingredient(s) in water with viscous material, such as natural or synthetic gums, resins, methylcellulose, sodium carboxymethylcellulose, or other well-known suspending agents.

In some embodiments, the administration of the compositions is oral administration. The compositions may be formulated for oral administration in any suitable form. For example, compositions for oral administration may be formulated in one or more of the following forms: a tablet, a troche, a powder, a granulate, a lozenge, a solution, a suspension, an emulsion, an elixir, a syrup, a wafer or a capsule filled with a solution, a suspension, an emulsion, an elixir, a syrup, a powder, a granulate, a tincture or a combination thereof. The compositions of the invention may be administered orally without further formulation. However, in some embodiments, oral forms of the compositions may comprise one or more pharmaceutically, veterinary and/or nutraceutically acceptable excipient(s). In some embodiments, the composition is a liquid oral composition, and may be in a form selected from a solution, suspension, an emulsion (optionally comprising an emulsifier), an elixir, a syrup, a tincture or a combination thereof. Liquid oral compositions are preferred for administration to non-human subjects.

The tablets, troches, pills, lozenges, capsules and the like may also contain any of the components as listed hereafter a binder such as acacia gum, corn starch or gelatin; excipients such as dicalcium phosphate, microcrystalline cellulose (MCC), silicified microcrystalline cellulose or powdered cellulose; a disintegrating agent such as corn starch, potato starch, alginic acid, alginate, polyvinyl pyrrolidone (PVP) and the like; a lubricant such as magnesium stearate; and a sweetening agent such a sucrose, lactose or saccharin may be added or a flavouring agent such as peppermint, oil of wintergreen, or cherry flavouring. When the dosage unit form is a capsule, it may contain, in addition to materials of the above type, a liquid carrier.

In some embodiments, the dosage unit form is a capsule. To form a capsule, typically the fatty acid component, stabiliser and any additional ingredient(s) are combined with one or more of the excipients (e.g. carriers) described herein to provide a solid or liquid formulation, which is then encased within a capsule shell. Any suitable capsule shell known in the art may be used, including hard and soft capsule shells. Suitable hard capsule shells may comprise gelatine, HPMC, starch, pullulan and/or polyvinyl acetate (PVA). Suitable soft capsules may comprise gelatin thickened with a thickening agent, such as a polyol (e.g. glycerine or sorbitol). As noted above, the capsule shell may be filled with any of the following dosage forms described herein: a solution, a suspension, an emulsion, an elixir, a syrup, a powder, a granulate or a combination thereof. When the capsule is filled with a solid dosage form, it may be dried prior to filling. In some embodiments, the solid dosage form is freeze-dried prior to filling the capsule shell. Alternatively, a liquid excipient may be added to provide a wet dosage form, such as a granulate, a solution, a suspension, an emulsion, an elixir or a syrup.

Various other materials may be present as coatings or to otherwise modify the physical form of the dosage unit. For instance, tablets, pills, or capsules may be coated with shellac, sugar or both. A syrup or elixir may contain the fatty acid component, stabiliser, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the fatty acid component and the stabiliser may be incorporated into sustained-release preparations and formulations.

Also included are preparations that are intended to be diluted, shortly before use, to liquid form preparations, such as for oral and/or sub-lingual administration. Such liquid forms include solutions, suspensions, and emulsions. These preparations may contain colorants, flavours, buffers, artificial and natural sweeteners, dispersants, thickeners, solubilising agents, and the like, in addition to the fatty acid component, stabiliser and any additional ingredient(s).

Formulations suitable for topical administration in the mouth (e.g. sub-lingual administration) include any liquid formulation described herein, preferably liquid formulations with a viscosity suitable for administration by dropper or syringe; lozenges comprising a flavoured base, usually sucrose and acacia or tragacanth; pastilles comprising an inert base such as gelatin and glycerin or sucrose and acacia; and mouthwashes comprising a suitable liquid carrier.

For topical administration to the epidermis the composition may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilising agents, dispersing agents, suspending agents, thickening agents, or colouring agents.

The pharmaceutical, veterinary and/or nutraceutical compositions may be formulated for parenteral administration (e.g. by injection, for example bolus injection or continuous infusion) and may be presented in unit dose form in ampoules, pre-filled syringes, small volume infusion or in multi-dose containers optionally with an added preservative. The compositions may take such forms as suspensions, solutions, or emulsions in oily or aqueous vehicles, and may contain formulation agents such as suspending, stabilising and/or dispersing agents.

Compositions for parenteral administration may also be provided in unit dosage form for ease of administration and uniformity of dosage. Unit dosage form as used herein refers to physically discrete units suited as unitary dosages for the subjects to be treated; each unit containing a predetermined quantity of active material calculated to produce the desired therapeutic effect in association with the required excipient.

Administration forms suitable for injectable use include sterile injectable solutions or dispersions, and sterile powders for the extemporaneous preparation of sterile injectable solutions. They should be stable under the conditions of manufacture and storage and may be preserved against oxidation and the contaminating action of microorganisms such as bacteria or fungi.

The solvent or dispersion medium for the injectable solution or dispersion may contain any of the conventional solvent or carrier systems, and may contain, for example, water, ethanol, polyol (for example, glycerol, propylene glycol and liquid polyethylene glycol, and the like), suitable mixtures thereof, and vegetable oils.

Sterile injectable solutions are prepared by incorporating the ingredients of the composition in the required amounts in the appropriate carrier with various other ingredients such as those enumerated above, as required, followed by sterilisation. Generally, dispersions are prepared by incorporating the various sterilised active ingredients into a sterile vehicle which contains the basic dispersion medium and the required other ingredients from those enumerated above. In the case of sterile powders for the preparation of sterile injectable solutions, preferred methods of preparation are vacuum drying or freeze-drying of a previously sterile suspension of the active ingredient plus any additional desired ingredients.

In some embodiments, the pharmaceutical compositions are provided in the form of a food product. Suitable food products include solid food products including baked goods and liquid food products such as a beverage. The composition may be incorporated into the food product during manufacture or may be added to an existing product. Therefore, the food products disclosed herein may comprise the fatty acid component, stabiliser and at least one further food ingredient.

Food ingredients may be any ingredient suitable for inclusion in a food product. When the food product is for human consumption, the food ingredient may be a Generally Recognised As Safe (GRAS) ingredient. The GRAS ingredient may be any ingredient included in the GRAS database maintained by the US Food and Drug Administration (FDA); and Flavor and Extract Manufacturers Association of the United States (FEMA) or other regulatory authorities in other geographical locations that assess general safety of food and feed ingredients.

In some embodiments, the food product is an animal treat, such as a biscuit or chew. The food product may be a functional food product, wherein the food product also comprises a pharmaceutically, veterinary and/or nutraceutically active ingredient. Any of the active pharmaceutical, veterinary and/or nutraceutical ingredients described above may be included in the functional food. The active pharmaceutical, veterinary and/or nutraceutical ingredient may be included as a part of the composition, or may be incorporated separately into the functional food product.

Each food ingredient may be present in a minimum amount of at least about 0.01%, 0.05%, 0.1% or 0.5%. Each food ingredient may be present in a maximum amount of up to about 25%, 22.5%, 20%, 18%, 15%, 10%, 8%, 5% or 1%. Each food ingredient may be included in an amount from any of these minimum amounts to any of these maximum amounts, for example, from about 0.01% to about 25% or about 0.5% to about 20%.

In some embodiments, the food product may comprise the composition in any amount sufficient to provide the composition to the subject upon consumption without unduly impacting the flavor of the food.

The minimum amount of composition may be at least about 0.1%, 0.5%, 1%, 2%, 3%, 4%, 5%, 6%, 7%, 8%, 9%, 10%, 11%, 12%, 13%, 14% or 15%. The maximum amount of composition may be not more than about 25%, 20%, 15%, 12% or 10%. The food product may comprise the composition in an amount from any of these minimum amounts to any of these maximum amounts provided the minimum amount is less than the maximum amounts, for example, from about 0.1% to about 25%, from about 10% to about 20%, or from about 5% to about 15%.

Pharmaceutically, veterinary and/or nutraceutically acceptable carriers and/or diluents include any and all solvents, dispersion media, coatings, antibacterial and antifungal agents, isotonic and absorption delaying agents and the like.

When desired, formulations adapted to give sustained release of the active ingredient(s) may be employed.

The practice of the present invention employs, unless otherwise indicated, conventional pharmaceutical and/or medical techniques within the skill of the art. Such techniques are well known to the skilled worker, and are explained fully in the literature.

Method of Treatment

The invention also provides a method of treating a subject suffering from or at risk of developing a skin disorder and/or an inflammatory condition, which may be due to a weakened immune system with or without the involvement of external parasites or other environmental or food allergens. The method comprises administering to a subject in need thereof an effective amount of the composition of the invention. Administration of the effective amount of the composition may thereby treat the skin disorder and/or inflammatory condition. Any of the compositions of the invention described herein may be employed in these methods.

Advantageously, administration of an effective amount of a composition of the invention may support the immune system and inflammatory defense mechanism of the subject. This may be advantageous for subjects suffering from or at risk of developing a skin disorder and/or an inflammatory condition.

The subject suffering from or at risk of developing a skin disorder may suffer from or be at risk of developing any disease, disorder or condition that affects the skin of a subject. In some embodiments, the skin disorder is caused by inflammation or inflammation is a symptom of the skin disorder. For example, the subject may suffer from or be at risk of developing a skin disorder selected from atopic dermatitis (including canine atopic dermatitis), contact dermatitis, dyshidrotic eczema, nummular dermatitis, seborrheic dermatitis and stasis dermatitis or a combination thereof.

The subject suffering from or at risk of developing an inflammatory condition may suffer from or be at risk of developing any form of inflammation whose treatment may be assisted by modulation of the endocannabinoid system. The inflammation may be localised or systemic.

In some embodiments, the inflammation may be a symptom or a cause a disease and/or disorder. The disease and/or disorder may be selected from inflammatory skin disorders, osteoarthrisis (OA), rheumatoid arthritis (RA), psoriatic arthritis (PsA), ankylosing spondylitis (AS), non-radiographic axial spondyloarthritis (nr-axSpA), allergic dermatitis including food and parasitic induced dermatitis, bacterial and fungal skin lesions, idiopathic arthritis, anterior knee pain, chilblains, chronic recurrent multifocal osteomyelitis, fibromyalgia, familial Mediterranean fever (FMF), gout, growing pains, haemochromatosic arthritis, localised scleroderma, lupus, polymyalgia rheumatica, reactive arthritis, ross river fever, scleroderma, Sever's disease, Sjogren's syndrome and spondyloarthritis, skin cancers or a combination thereof.

In some embodiments, the skin disorder and/or inflammatory condition is an inflammatory skin disorder. The inflammatory skin disorder may be associated with immunoglobulin E (IgE) mediated type 1 hypersensitivity (allergic) responses. In some embodiments, the inflammatory skin disorder includes atopic dermatitis (such as canine atopic dermatitis), flea bite hypersensitivity, and food hypersensitivity.

In some embodiments, the skin disorder and/or inflammatory condition is associated with elevated levels of a biomarker selected from MCP-1, IL-8, KC, CXCL1, HGF and RAGE or a combination thereof. The methods may therefore further comprise a step of assessing the level of one or more of these biomarkers prior and/or following administration of the composition. In some embodiments, administration of the composition is effective to lower the level of one or more of these biomarkers compared to its pre-treatment level.

A symptom of a skin disorder and/or inflammation may be pain. The pain may be noiciceptive pain, psychogenic pain and/or neuropathic pain. Noiciceptive pain is associated with stimulation of sensory nerve endings (or noiciceptors). Psychogenic pain is associated with psychological factors resulting in a pain disorder (often diagnosed when other physical causes for pain are ruled out). Neuropathic pain is associated with damage or malfunction of the peripheral nervous system (PNS) or central nervous system (CNS). Cannabinoid receptors (e.g. CB1 and CB2 receptors) have been reported as being expressed in the PNS and CNS. The compositions of the invention therefore may be used in the treatment of any or all forms of pain associated with a skin disorder and/or inflammation.

The method comprises administering an effective amount of the composition. The effective amount may be determined by the skilled person based on numerous factors, including the severity and kind of symptoms of the subject, the subject's medical history, the subject's physical attributes (weight, sex, etc), the specific combination of active ingredients included in the pharmaceutical compositions to be administered and the administration route.

The administration of the composition is preferably oral administration. Oral administration is typically considered systemic administration. Therefore, oral administration is preferred to treat a generalised skin disorder and/or generalised inflammation, or a skin disorder and/or inflammation experienced at multiple locations by the subject. Further, an advantage of the composition of the invention is that the active ingredients are orally bioavailable.

In some embodiments, the method comprises oral administration of the composition. The composition may be administered at any suitable frequency. In some embodiments, the composition is administered once or twice daily. Twice daily administration typically involves administration of equivalent doses of the composition at least about 6 h apart. The administration may be without food, or may be with food. Administration of the composition of the invention with food typically involves administering the composition within about 1 h after the subject has consumed food. It is believed that fats may assist oral absorption of CBD, such as fats consumed in food, or contained in the fatty acid component of the compositions of the invention.

The method may also comprise administering a further active ingredient. This active ingredient may be administered simultaneously, separately or consecutively with the composition. By simultaneously it is meant that each of the composition and the further active ingredient are administered at the same time in the same pharmaceutical composition. By separately it is meant that each of the composition and the further active ingredient are administered at the same time in different compositions and optionally by different routes of administration. By consecutively it is meant that each of the composition and the further active ingredient are administered separately optionally by different administration mutes and may be at different times. Typically, when the composition and the other active ingredient are administered consecutively they are administered within 24 hours, or within 12, 8, 6, 5, 4, 3, 2, or 1 hour(s) of each other. The composition may be administered before or after the other active ingredient. Further, the route of administration for the composition and the other active ingredient may be the same or different.

The further active ingredient may be any of the further active ingredients disclosed herein. In some embodiments, the further active ingredient is an ingredient used in the management and/or treatment of a skin disorder and/or inflammation or a symptom thereof. In some embodiments, the further active ingredient is selected from oclacitinib (eg Apoquel™), a JAK inhibitor (e.g. a JAK3 inhibitor), a monoclonal antibody targeting treatment of a skin disorder and/or inflammation, a corticosteroid or a combination thereof. In some embodiments, the composition of the invention may be used as an adjunct therapy with the further active ingredient.

The subject mentioned above may be any subject with an endocannabinoid system. Therefore, the subject may be a mammal, including a human and also including non-human species. The non-human species include but are not limited to companion animals. Companion animals include dogs, cats, guinea pigs, hamsters, horses, cattle, goats, sheep and the like. Typically, the subject is a dog or cat, most typically a dog. However, such animals in need of such treatment may also include zoo animals such as monkeys, elephants, giraffes and other ungulates, bears, mice and other small mammals.

In some embodiments, the method may be a method of treating a skin disorder and/or an inflammatory condition, comprising administering an effective amount to a subject in need thereof. In some embodiments, the subject is a companion animal, preferably a dog. In some embodiments, the skin disorder and/or inflammatory condition is CAD.

Also disclosed herein is the use of one or more of cannabidiol, an omega-3 fatty acid, an omega-6 fatty acid and/or a sterol in the preparation of a pharmaceutical, veterinary and/or nutraceutical composition comprising cannabidiol, an omega-3 fatty acid, an omega-6 fatty acid and a sterol. The nutraceutical, veterinary and/or nutraceutical composition may be any of the compositions described herein and may be for use in any of the methods described herein.

### EXAMPLES

The invention will be further described by way of non-limiting example(s). It will be understood to persons skilled in the art of the invention that many modifications may be made without departing from the spirit and scope of the invention.

### Example 1

The compositions of formulations IVP1 and IVP2 are set out in Table 1 below. The components were mixed together at the amounts indicated. All components of IVP1 and IVP2 were mixed at ambient temperature (˜25° C.).

TABLE 1
Compositions IVP1 and IVP2

| | | IVP2-DermaCann whole |
|---|---|---|
| | IVP1-DermaCann isolate | hemp extract (DC_WHE) |
| Ingredient | (DC_ISO) (g/100 ml) | (g/100 ml) |
| Hemp oil^{a} | 46.61 | 46.61 |
| Flax seed oil^{b} | 46.61 | 46.61 |
| Cannabidiol (CBD)^{c} | 0.5 | — |
| CBD-rich Cannabis extract^{d} | — | 0.5 |
| Antioxidant (ParaMega ™) | 0.2 | 0.2 |
| α-Pinene | 0.075 | 0.075 |
| β-Caryophyllene | 0.3 | 0.3 |
| α-Tocopherol (vitamin E) | 5.4 | 5.4 |
| β-Sitosterol | 0.3 | 0.3 |

Notes:
^{a}The fatty acid composition of the hemp seed oil used was 4-10% palmitic acid (16:0), 1-4% stearic acid (18:0), 6-20% oleic acid (18:1), 45-65% linoleic acid (C18:2), 14-28% α-linolenic acid (18:3), 1-5% γ-linolenic acid (18:3) and 0-2.5% stearidonic acid (18:4);
^{b}The fatty acid composition of the flax seed oil used was 3-8% palmitic acid (16:0), 2-8% stearic acid (18:0), 11-24% oleic acid (18:1), 12-20% linoleic acid (18:2) and 50-65% α-linolenic acid (18:3);
^{c}The CBD used is 99.8% pure based on a High-Performance Liquid Chromatographic (HPLC) analysis. The CBD used comprises not more than 0.1% Δ⁹-tetrahydrocannabinol (THC), not more than 0.5% cannabidivirin (CBDV), not more than 0.5% CBD-C4 and not more than 0.1% other phytocannabinoids.
^{d}CBD-rich plant extract comprises 10.5% CBD in hemp seed oil and not more than 0.2% THC and not more than 1% water.

### Example 2

This Example describes a double-blinded placebo-controlled randomised trial of formulations described in Example 1 involving 13 dogs, approximately balanced by sex, of various breeds and ages with Canine Atopic Dermatitis (CAD) that have been already diagnosed and controlled to some extent but not symptom free.

2.1 Subjects

Dogs (Canis familiaris) of any breed or gender, including neutered animals, presented to dermatology specialists that otherwise met the inclusion and exclusion criteria.

Inclusion criteria. Dogs included were (a) ≥5 kg and ≤45 kg in weight; (b) >6 months of age; (c) previously diagnosed with atopic dermatitis by clinical and historical findings and exclusion of other potential causes relevant to that patient; (d) experience at least “Mild” itching on the enhanced pruritus scale (Hill et al, 2007) and/or a minimum score of approximately 10 on the CADESI-4 score; (e) no changes in any atopy medication for 7 days prior to Day 0; (f) no change in any atopy medication during the 56 (+5) days of the study; (g) no change in Cyclosporin dose for 4 weeks prior to enrolment; (h) no change to cyclosporin dose (if receiving) throughout the study period; (i) no administration of Prednisolone for 8 weeks prior to enrolment; (j) if they have been on allergen immunotherapy, it has been administered for at least 12 months prior to enrolment in the study; (k) during the course of the study dose, and frequency of allergen immunotherapy will have no change to the established programme; (l) dogs have lived in their current residence for >1 month; (m) dogs are treated with an isoxaline or other effective flea treatment and remain treated throughout the study period; (n) dogs are fed a consistent diet with no additional Omega 3 and 6 supplementation or high level vitamin E supplementation for at least one month prior to enrolment; (o) dogs receive no omega 3 or 6 and vitamin E supplements additional to that found within the diet during the 56 (+/−2) days of the trial; (p) only one dog from a single household may be enrolled. If more than one animal has atopy the animal chosen must meet the inclusion criteria and deemed as the most compliant for administering medications; (q) in multiple pet households the enrolled animal must be treated/fed separately throughout the study; and (r) households with other flea harbouring pets are eligible for enrolment provided all animals are treated with effective flea control and the study animal(s) are fed/medicated separately.

Participating dogs have already been on an elimination diet (for possible food allergies) and the dogs are on a reasonably stable treatment regime (treatments listed by subject). Dogs are fed the same recorded type and amount of diet prior to and during treatment.

The total amounts of omega-3 and omega-6 fatty acids, vitamin E received before and during the trial are calculated for each dog using the manufacturer's information of the fatty acid content of the commercial diet and adding the fatty acid and vitamin E content of the administered supplement.

Dogs with CAD are divided into the following three groups and administered a control veterinary product (CVP) or one of two investigational veterinary products (IVPs). Neither owner nor clinician was aware of the nature of the supplementation.
A. Treatment group A (CVP placebo group—5 dogs) medium chain triglyceride oil (MCT) oil (coloured with chlorophyll) 1 ml/10 kg twice daily within 1 h of food for eight weeks.
   a. If this dose is considered too high for a dog due to potential laxative effect, dose is substituted for high heat canola oil or grape seed oil.
B. Treatment group B (IVP1 group—4 dogs) receive IVP1 (DermaCann_isolate; DC_ISO) as described in Example 1 at a dose of 1 ml/10 kg twice daily within 1 h of food for eight weeks.
C. Treatment group C (IVP2 group—4 dogs) receive IVP2 (DermaCann Whole Hemp Extract; DC_WHE) as described in Example 1 at a dose of 1 ml/10 kg twice daily within 1 h of food for eight weeks

2.2 Treatment protocol. Treatment was administered twice daily (approximately every 12 hours) by oral dosing using syringes or directly on to the dog's food with observed eating. Syringes fit into an adaptor attached to the top of the product bottles.

Dogs were dosed within 60 minutes of being fed a regular size meal (ie after a meal). To ensure accurate dosing, the treatment was given directly into the dog's mouth. The syringe containing the IVP/CVP was placed toward the back of the mouth (over the base of the tongue if possible) and the contents expelled in a manner to encourage swallowing. If the dog refuses oral medication, the dose was applied to a small amount of food (treat size) and the owner supervised that the entire amount of food containing the dose was consumed by the dog.

Dose Calculation. The IVP/CVP dose was calculated based on individual animal bodyweights at Day 0 according to the treatment tables below (Tables 2 and 3).

TABLE 2
Treatment Regime

| Treatment | Dose | | | Treatment | Number of |
|---|---|---|---|---|---|
| Group | Rate | Route | Frequency | Day | Animals |
| A (placebo) | 1 mL/10 kg | oral | Twice daily at | 0-56 (+5 days) | 5 |
| B | 1 mL/10 kg | | approximately 12 | 0-56 (+5 days) | 4 |
| (DermaCann_ | | | hourly intervals | | |
| isolate) | | | | | |
| C | 1 mL/10 kg | | | 0-56 (+5 days) | 4 |
| (DermaCann_ | | | | | |
| whole hemp | | | | | |
| extract) | | | | | |

TABLE 3
Dose Chart

| Dog Weight in KG | | mLs per dose | mLs per day |
|---|---|---|---|
| 5.0 | kg | 0.50 | 1.00 |
| 5.1-10 | kg | 1.00 | 2.00 |
| 10.1-15 | kg | 1.50 | 3.00 |
| 15.1-20.0 | kg | 2.00 | 4.00 |
| 20.1-25 | kg | 2.50 | 5.00 |
| 25.1-30.0 | kg | 3.00 | 6.00 |
| 30.1-35 | kg | 3.50 | 7.00 |
| 35.1-40 | kg | 4.00 | 8.00 |
| 40.1-45 | kg | 4.50 | 9.00 |

2.3 Preliminary Safety Assessment and Adverse Events

All 3 treatment groups, ie including both IVP groups, reported very few adverse events, no repeat adverse events in any individual subject, all adverse events were mild and were resolved. No dogs were withdrawn from the trial as a result of any adverse events. Adverse events were distributed between the 3 treatment groups, including control. There were twice as many dogs (4 dogs, 6 AEs) in group A (placebo) experiencing AEs than either treatment groups B (2 dogs; 3 AEs) or C (2 dogs; 6 AEs). Within the treatment groups, all AEs were reported as mild, with the exception of a single moderate AE in group B that was determined to be unrelated to IVP administration. No dogs experiencing AEs were removed from the study. The low number and severity of these adverse events is strongly indicative that the 2 IVP treatments are well tolerated.

2.4 Clinical Examination

Clinical lesions are scored using the Canine Atopic Dermatitis Extent and Severity Index (CADESI-4):
a. A threshold value is set as an inclusion criteria for each dog in the study.
b. The CADESI-4 scoring system of assessment is used at the inclusion date, after 3 or 4 weeks of treatment and after 6 or 8 weeks of treatment.

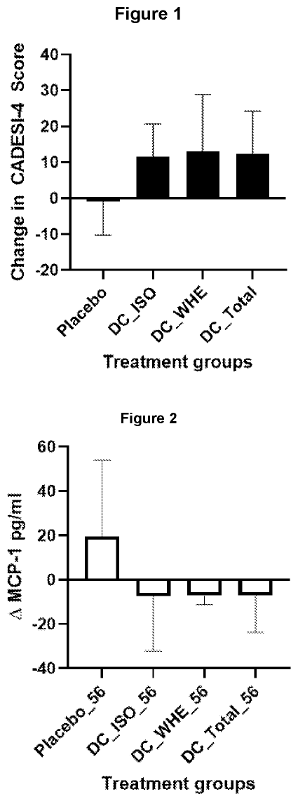

Results are summarized in the Table 4 and FIG. 1. FIG. 1 was prepared by comparing the Total CADESI-4 score differences between Day 0 and day 58 for all dogs in each treatment group (placebo, DC_ISO and DC_WHE) with scores for dogs treated with either IVP formulation (DC_total). Thus, DC_total represents the difference in CADESI score for all dogs in both the DC_ISO and DC_WHE treatment groups. The difference in CADESI scores for each dog represents the reduction in erythema, lichenification, excoriation and alopecia seen in these atopic dogs over the duration of the study. Normality of the data was assessed using Shapiro-Wilk test and all groups were found to pass the normality test. Data for DC_ISO and DC_WHE were compared using t-test and found to not be significantly different (P=0.88). Data for both treatments was then pooled and compared with placebo using t-test (P=0.06) which was considered significant at the P<0.1 level as is accepted in initial efficacy studies.

TABLE 4
CADESI-4 results

| | Days after | | CADESI 4 | CADESI Diff |
|---|---|---|---|---|
| Dog ID | treatment | Treatment | TOTAL | Days 0-56 |
| 1 | 0 | C | 16 | −4 |
| 1 | 28 | C | 15 | |
| 1 | 56 | C | 20 | |
| 2 | 0 | B | 23 | 5 |
| 2 | 28 | B | 16 | |
| 2 | 56 | B | 18 | |
| 3 | 0 | A | 57 | −8 |
| 3 | 28 | A | 64 | |
| 3 | 56 | A | 65 | |
| 4 | 0 | B | 21 | 9 |
| 4 | 28 | B | 14 | |
| 4 | 56 | B | 12 | |
| 5 | 0 | C | 19 | 14 |
| 5 | 28 | C | 16 | |
| 5 | 56 | C | 5 | |
| 6 | 0 | A | 31 | −9 |
| 6 | 28 | A | 22 | |
| 6 | 56 | A | 40 | |
| 7 | 0 | A | 9 | −6 |
| 7 | 28 | A | 6 | |
| 7 | 56 | A | 15 | |
| 8 | 0 | B | 35 | 25 |
| 8 | 28 | B | 25 | |
| 8 | 56 | B | 10 | |
| 9 | 0 | C | 12 | 8 |
| 9 | 28 | C | 10 | |
| 9 | 56 | C | 4 | |
| 10 | 0 | B | 9 | 7 |
| 10 | 28 | B | 3 | |
| 10 | 56 | B | 2 | |
| 11 | 0 | A | 17 | 10 |
| 11 | 28 | A | 15 | |
| 11 | 56 | A | 7 | |
| 12 | 0 | C | 58 | 34 |
| 12 | 28 | C | 26 | |
| 12 | 56 | C | 24 | |
| 13 | 0 | A | 19 | 9 |
| 13 | 28 | A | 21 | |
| 13 | 56 | A | 10 | |

2.5 Blood Testing

On a 3 or 4 weekly basis during the trial period blood samples were collected from each subject into lithium heparin tubes, centrifuged at 1500×g for 15 minutes at 4° C. Plasma was aliquoted, immediately frozen and kept until analysed.

Haematological and biochemical blood tests are done to monitor for possible concomitant diseases at the start and end of the study and/or reasons for dog exclusion.
Serum samples are assayed on the day of blood sampling to determine creatinine, urea, alanine aminotransferase (ALT) and alkaline phosphatase (ALP).
EDTA blood samples for complete blood count (CBC) and white cell differential count (WCDC) determinations within 1-3 hours of sampling.

2.6 Statistics
Descriptive statistics is used to describe the basic features of the data.
Shapiro-Wilk test is performed to examine whether the data are normally distributed
Non-parametric Mann-Whitney U and Kolmogorov-Smirnov tests are used to check for statistically significant differences between results obtained in the treatment and placebo groups if the data is not normally distributed
ANOVA and T-tests are used if the data is normally distributed.
Subjective evaluations of pruritus is performed by continuous scale plotting quantitated and the scale distance evaluations with continuous data for normality of distribution by the Kolmogorov-Smimov or Shapiro-Wilks test.
Behaviour is assessed using categorical data analysis.
Clinician and owner scores within groups is evaluated using by a Wilcoxon matched pair test.
Results differ significantly at p<0.1, p<0.15 are indicative.

2.7 Gene Expression Array

Samples of whole blood were taken from each dog at 0, 28 and 56 days after treatment and stored in an RNA protect tube at −20° C. until analysed. RNA was extracted using the Rneasy Animal Blood kit following the Protocol: Purification of total RNA>200 Nucleotides (Excluding miRNA) from RNAprotect Stabilized Animal Blood. A custom prepared array of pain and inflammatory genes that had been previously used in healthy dogs treated single doses of cannabinoids was used to assess the genes in 3 dogs comparing pretreatment expression with expression at day 56 after treatment.

The three dogs selected for gene expression analysis included one that was a dog with no clinical response in the placebo group and the other two dogs that were observed to be clinical responders in each of the VP treatment groups. Based on the above screen, the expression of several genes was regulated in a biologically important manner. The results of this screen are summarised in Table 5 and Table 6 provides additional information for each of the selected genes.

Each gene assessment represents the fold change (up regulation is a positive value and down regulation is a negative value) for each dog.

Fold-regulation represents fold-change results in a biologically meaningful way. Fold-change values greater than one indicate a positive- or an up-regulation, and the fold-regulation is equal to the fold-change.

Fold-change values less than one indicate a negative or down-regulation, and the fold-regulation is the negative inverse of the fold-change.

TABLE 5
Results of gene regulation screen

| | FOLD CHANGE | | FOLD CHANGE | | FOLD CHANGE | |
|---|---|---|---|---|---|---|
| | Dog 8, group B | | Dog 6; group A | | Dog 5 group C | |
| | (DermaCann ISO) | | (PLACEBO) | | (DermaCann WHE) | |
| Gene | Fold | Fold | Fold | Fold | Fold | Fold |
| Symbol | Change | Regulation | Change | Regulation | Change | Regulation |
| ATF3 | 0.806641759 | −1.2397077 | 0.20877198 | −4.790 | 1.879 | 1.879 |
| CCL4 | 0.721964598 | −1.385109468 | 4.75682846 | 4.757 | 2.990 | 2.990 |
| CTLA4 | 2.907945035 | 2.907945035 | 1.558329159 | 1.558 | 2.567 | 2.567 |
| CXCL8 | 0.517632462 | −1.931872658 | 1.00695555 | 1.007 | 0.374 | −2.676 |
| IL1A | 0.823591017 | −1.214194884 | 1.474269217 | 1.474 | 0.358 | −2.789 |

TABLE 6
Description of selected genes included in custom array

| Gene | | | | Catalog |
|---|---|---|---|---|
| Symbol | Alias | Refseq # | Full Name | Number |
| ATF3 | LOC612911 | XM_847382 | activating transcription | PPF13829A |
| | | | factor 3 | |
| CCL4 | CCL4, CC chemokine | NM_001005250 | chemokine (C-C motif) | PPF00667A |
| | ligand 4, chemokine C-C | | ligand 4 | |
| | motif ligand 4, C-C | | | |
| | motif chemokine 4, | | | |
| | small-inducible cytokine | | | |
| | A4, MIP-1B | | | |
| CTLA4 | CTLA4, CD152, CTLA-4, | NM_001003106 | cytotoxic T- | PPF00272A |
| | RPL19, cytotoxic T- | | lymphocyte-associated | |
| | lymphocyte protein 4, | | protein 4 | |
| | cytotoxic T-lymphocyte- | | | |
| | associated antigen 4, | | | |
| | costimulatory molecule | | | |
| | B7 receptor CD152 | | | |
| CXCL8 | IL8, interleukin-8, IL-8, | NM_001003200 | interleukin 8 | PPF00414A |
| | C-X-C motif chemokine | | | |
| | 8, chemokine (C-X-C | | | |
| | motif) ligand 8 | | | |
| IL1A | IL1A, interleukin-1 | NM_001003157 | interleukin 1, alpha | PPF00350A |
| | alpha, IL-1 alpha | | | |

2.8 Canine Milliplex Cytokine/Chemokine Array

The plasma samples were collected (see section 2.5 above) and stored at −20° C. until assayed using the Milliplex Canine Cytokine/Chemokine Magnetic Bead Panel—Immunology Multiplex Array to determine cytokine and chemokine production of the dogs following oil exposure. Samples were assayed as collected as well as 2 times concentrated to maximise the chance of detecting these biomarkers in the dog plasma.

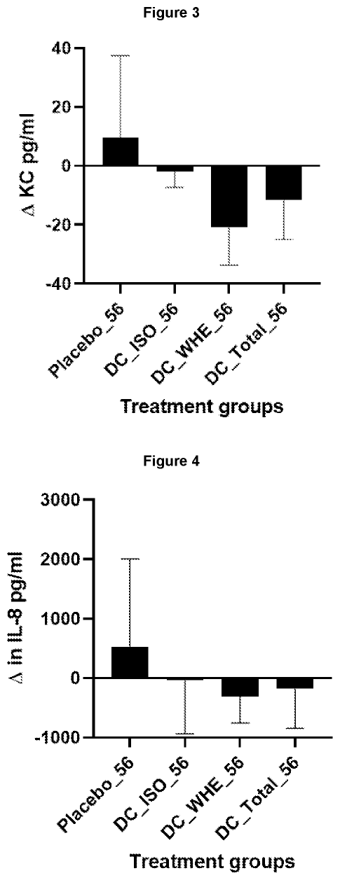

Analytes in the Canine Milliplex Cytokine/Chemokine panel include GM-CSF, IFN-γ, IL-2, IL-6, IL-7, IL-8, IL-10, IL-15, IL-18, IP-10, KC-like, MCP-A and TNF-α. Results are shown in Table 7 and FIGS. 2-4. These results show a clear difference in MCP-A, IL-8 and KC concentrations for both treatment groups relative to the placebo group.

TABLE 7
Results of Milliplex Cytokine/Chemokine panel

| Dog | Treatment | | IL-8 | Δ-IL-8 | MCP-1 | Δ-MCP- | KC | Δ- |
|---|---|---|---|---|---|---|---|---|
| ID | group | Visit | conc | conc | conc | 1 conc | conc | KC_conc |
| 1 | C | 0 Days | 41.98 | −27.01 | 40.83 | 0 | 37.94 | −30.06 |
| 1 | C | 28 | 14.97 | −27.01 | 40.83 | −4.9 | 7.88 | −33.675 |
| | | Days | | | | | | |
| 1 | C | 56 | 14.97 | | 35.93 | | 4.265 | |
| | | Days | | | | | | |
| 2 | B | 0 Days | 1630 | 5893.5 | 49.215 | −13.285 | 27.5 | 101.105 |
| 2 | B | 28 | 7523.5 | −1100 | 35.93 | −18.86 | 128.605 | −9.84 |
| | | Days | | | | | | |
| 2 | B | 56 | 530 | | 30.355 | | 17.66 | |
| | | Days | | | | | | |
| 3 | A | 0 Days | 365.59 | −134.17 | 30.355 | 95.5 | 3.35 | 13.105 |
| 3 | A | 28 | 231.42 | 1322.41 | 125.855 | 32.12 | 16.455 | 47.66 |
| | | Days | | | | | | |
| 3 | A | 56 | 1688 | | 62.475 | | 51.01 | |
| | | Days | | | | | | |
| 4 | B | 0 Days | 842.5 | −560.84 | 30.355 | −6.52 | 3.07 | 0 |
| 4 | B | 28 | 281.66 | −252.5 | 23.835 | 12.725 | 3.07 | 0 |
| | | Days | | | | | | |
| 4 | B | 56 | 590 | | 43.08 | | 3.07 | |
| | | Days | | | | | | |
| 5 | C | 0 Days | 687 | −348.35 | 77.585 | −4.595 | 37.12 | 3.035 |
| 5 | C | 28 | 338.65 | −474.695 | 72.99 | −12.28 | 40.155 | −24.78 |
| | | Days | | | | | | |
| 5 | C | 56 | 212.305 | | 65.305 | | 12.34 | |
| | | Days | | | | | | |
| 6 | A | 0 Days | 2075.5 | −1552 | 56.295 | −11.075 | 45.66 | −11.375 |
| 6 | A | 28 | 523.5 | −1571.5 | 45.22 | 11.695 | 34.285 | −26.155 |
| | | Days | | | | | | |
| 6 | A | 56 | 504 | | 67.99 | | 19.505 | |
| | | Days | | | | | | |
| 7 | A | 0 Days | 221.28 | 237.735 | 62.475 | −9.585 | 10.925 | 1.835 |
| 7 | A | 28 | 459.015 | 55.59 | 52.89 | −21.645 | 12.76 | 24.43 |
| | | Days | | | | | | |
| 7 | A | 56 | 276.87 | | 40.83 | | 35.355 | |
| | | Days | | | | | | |
| 8 | B | 0 Days | 940 | −298 | 52.89 | −22.535 | 12.27 | −8.31 |
| 8 | B | 28 | 642 | 130 | 30.355 | −36.98 | 3.96 | 0.905 |
| | | Days | | | | | | |
| 8 | B | 56 | 1070 | | 15.91 | | 13.175 | |
| | | Days | | | | | | |
| 9 | C | 0 Days | 1127.5 | −725.745 | 49.215 | 16.09 | 24.635 | 19.085 |
| 9 | C | 28 | 401.755 | −856.645 | 65.305 | −8.385 | 43.72 | −21.565 |
| | | Days | | | | | | |
| 9 | C | 56 | 270.855 | | 40.83 | | 3.07 | |
| | | Days | | | | | | |
| 10 | B | 0 Days | 354.435 | −14.65 | 116.4 | 19.98 | 3.195 | 3.345 |
| 10 | B | 28 | 339.785 | 1056.565 | 136.38 | 13.815 | 6.54 | 1.07 |
| | | Days | | | | | | |
| 10 | B | 56 | 1411 | | 130.215 | | 4.265 | |
| | | Days | | | | | | |
| 11 | A | 0 Days | 295.89 | −49.48 | 123.015 | 6.13 | 5.03 | 3.44 |
| 11 | A | 28 | 246.41 | 2387.61 | 129.145 | 70.54 | 8.47 | 2.255 |
| | | Days | | | | | | |
| 11 | A | 56 | 2683.5 | | 193.555 | | 7.285 | |
| | | Days | | | | | | |
| 12 | C | 0 Days | 419.485 | −287.89 | 62.475 | −7.855 | 6.39 | 23.44 |
| 12 | C | 28 | 131.595 | 111.015 | 54.62 | −2.995 | 29.83 | −3.32 |
| | | Days | | | | | | |
| 12 | C | 56 | 530.5 | | 59.48 | | 3.07 | |
| | | Days | | | | | | |
| 13 | A | 0 Days | 118.195 | 990.805 | 105.675 | −16.105 | 3.07 | 3.02 |
| 13 | A | 28 | 1109 | 437.805 | 89.57 | 5.55 | 6.09 | 0 |
| | | Days | | | | | | |
| 13 | A | 56 | 556 | | 111.225 | | 3.07 | |
| | | Days | | | | | | |

2.9 ELISA Assay

The samples obtained at section 2.5 above were also subjected to RayBio ELISA assays, which detected significant (p=0.05) differences in concentrations of Chemokine (C-X-C motif) ligand 1 (CXCL1), Hepatocyte Growth Factor (HGF), interferon-α (IFN-α/IFN-a), Receptor for Advanced Glycation End products (RAGE), resistin and interleukin-1 (IL-1) proteins between placebo and at least one of the treatment groups. The changes in concentration of CXCL1, HGF and RAGE correlate to improvements in skin disorders and inflammatory conditions, such as atopic dermatitis, and modulation of the immune system.

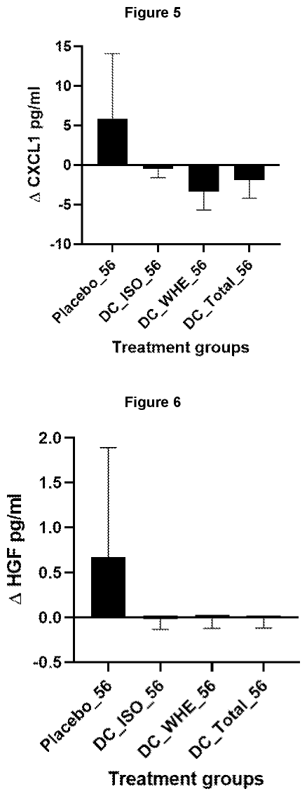

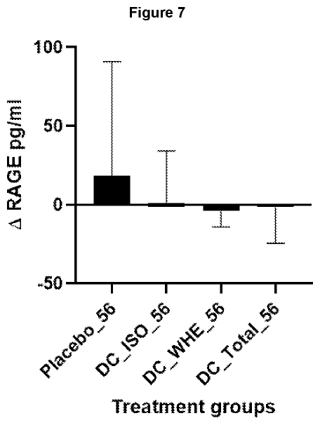

The results of the ELISA assay are summarized in Table 8 and FIGS. 5-7.

TABLE 8
Results of RayBio ELISA assay

| | Treat- | | | | | | | | | | | | | |
|---|---|---|---|---|---|---|---|---|---|---|---|---|---|---|
| Dog | ment | | | Δ- | | Δ- | | Δ- | | Δ- | | Δ- | | Δ- |
| ID | group | Visit | CXCL1 | CXCL1 | HGF | HGF | IFN-a | IFN-a | RAGE | RAGE | IL1 | IL1 | Resistin | Resistin |
| 3 | A | 0 | 2.018 | 10.659 | 0.235 | 0.564 | 2.066 | −0.007 | 31.925 | 136.977 | 334.844 | 4.7 | 230.763 | −82.504 |
| | | Days | | | | | | | | | | | | |
| 3 | A | 28 | 12.677 | 19.602 | 0.799 | 0.818 | 2.059 | 0.484 | 168.902 | 145.384 | 339.544 | 0 | 148.259 | 96.446 |
| | | Days | | | | | | | | | | | | |
| 3 | A | 56 | 21.62 | | 1.053 | | 2.55 | | 177.309 | | 334.844 | | 327.209 | |
| | | Days | | | | | | | | | | | | |
| 6 | A | 0 | 5.737 | 1.401 | 0.112 | 0.012 | 0.002 | 0.048 | 56.023 | −11.262 | 2.41 | 3.784 | 660.878 | −215.625 |
| | | Days | | | | | | | | | | | | |
| 6 | A | 28 | 7.138 | −0.5 | 0.124 | −0.045 | 0.05 | 0 | 44.761 | −2.163 | 6.194 | 3.784 | 445.253 | −30.519 |
| | | Days | | | | | | | | | | | | |
| 6 | A | 56 | 5.237 | | 0.067 | | 0.002 | | 53.86 | | 6.194 | | 630.359 | |
| | | Days | | | | | | | | | | | | |
| 7 | A | 0 | 0.054 | 0.799 | 0.112 | −0.045 | 0.08 | −0.078 | 90.455 | 4.165 | 10.035 | −7.625 | 316.596 | 23.848 |
| | | Days | | | | | | | | | | | | |
| 7 | A | 28 | 0.853 | 7.11 | 0.067 | 0.019 | 0.002 | −0.047 | 94.62 | −3.172 | 2.41 | 0 | 340.444 | −10.906 |
| | | Days | | | | | | | | | | | | |
| 7 | A | 56 | 7.164 | | 0.131 | | 0.033 | | 87.283 | | 10.035 | | 305.69 | |
| | | Days | | | | | | | | | | | | |
| 11 | A | 0 | 0.054 | 4.208 | 0.503 | 1.104 | 1.573 | 0.306 | 154.809 | 85.572 | 107.619 | 8.441 | 85.683 | 1082.635 |
| | | Days | | | | | | | | | | | | |
| 11 | A | 28 | 4.262 | 2.837 | 1.607 | 2.743 | 1.879 | −0.831 | 240.381 | −9.85 | 116.06 | −61.92 | 1168.318 | 615.766 |
| | | Days | | | | | | | | | | | | |
| 11 | A | 56 | 2.891 | | 3.246 | | 0.742 | | 144.959 | | 45.699 | | 701.449 | |
| | | Days | | | | | | | | | | | | |
| 13 | A | 0 | 0.054 | 0 | 1.768 | −0.327 | 0.2 | −0.033 | 218.919 | −8.204 | 25.696 | 24.047 | 271.22 | −122.961 |
| | | Days | | | | | | | | | | | | |
| 13 | A | 28 | 0.054 | 0 | 1.441 | −0.197 | 0.167 | 0.011 | 210.715 | −38.15 | 49.743 | 11.955 | 148.259 | −221.007 |
| | | Days | | | | | | | | | | | | |
| 13 | A | 56 | 0.054 | | 1.571 | | 0.211 | | 180.769 | | 37.651 | | 50.213 | |
| | | Days | | | | | | | | | | | | |
| 2 | B | 0 | 4.095 | 17.228 | 0.124 | 0.013 | 0.002 | 0 | 16 | 31.112 | 10.035 | 27.616 | 489.314 | 64.37 |
| | | Days | | | | | | | | | | | | |
| 2 | B | 28 | 21.323 | −1.753 | 0.137 | 0.056 | 0.002 | 0 | 47.112 | 38.296 | 37.651 | 15.661 | 553.684 | −138.306 |
| | | Days | | | | | | | | | | | | |
| 2 | B | 56 | 2.342 | | 0.18 | | 0.002 | | 54.296 | | 25.696 | | 351.008 | |
| | | Days | | | | | | | | | | | | |
| 4 | B | 0 | 1.068 | 0.255 | 0.356 | 0.047 | 0.012 | 0.095 | 53.86 | 12.072 | 17.817 | 0 | 198.053 | −61.006 |
| | | Days | | | | | | | | | | | | |
| 4 | B | 28 | 1.323 | −1.01 | 0.404 | −0.13 | 0.1082 | 0.0217 | 65.932 | 17.557 | 17.817 | −11.623 | 137.047 | −69.456 |
| | | Days | | | | | | | | | | | | |
| 4 | B | 56 | 0.054 | | 0.174 | | 0.033 | | 71.417 | | 6.194 | | 128.597 | |
| | | Days | | | | | | | | | | | | |
| 8 | B | 0 | 2.518 | −1.826 | 0.521 | −0.035 | 0.834 | 0.347 | 99.057 | −10.006 | 10.035 | 15.661 | 217.174 | 157.53 |
| | | Days | | | | | | | | | | | | |
| 8 | B | 28 | 0.692 | 0.836 | 0.486 | −0.006 | 1.181 | −0.36 | 89.051 | −35.943 | 25.696 | 0 | 374.704 | 372.363 |
| | | Days | | | | | | | | | | | | |
| 8 | B | 56 | 3.354 | | 0.515 | | 0.474 | | 63.114 | | 10.035 | | 589.537 | |
| | | Days | | | | | | | | | | | | |
| 10 | B | 0 | 0.054 | 0 | 0.149 | 0.044 | 0.002 | 0.251 | 31.925 | 71.164 | 1 | 16.817 | 142.661 | 44.409 |
| | | Days | | | | | | | | | | | | |
| 10 | B | 28 | 0.054 | 0 | 0.193 | 0.068 | 0.253 | 0 | 103.089 | −15.925 | 17.817 | 1.41 | 187.07 | −56.978 |
| | | Days | | | | | | | | | | | | |
| 10 | B | 56 | 0.054 | | 0.217 | | 0.002 | | 16 | | 2.41 | | 85.683 | |
| | | Days | | | | | | | | | | | | |
| 1 | C | 0 | 5.445 | −5.391 | 0.825 | 0.07 | 3.559 | 2.205 | 115.196 | 17.286 | 33.65 | −3.986 | 2048.554 | 368.973 |
| | | Days | | | | | | | | | | | | |
| 1 | C | 28 | 0.054 | −5.391 | 0.895 | 0.029 | 5.764 | 0.726 | 132.482 | −2.904 | 29.664 | 4.001 | 2417.527 | −382.215 |
| | | Days | | | | | | | | | | | | |
| 1 | C | 56 | 0.054 | | 0.854 | | 4.285 | | 112.292 | | 37.651 | | 1666.339 | |
| | | Days | | | | | | | | | | | | |
| 5 | C | 0 | 6.135 | 1.751 | 0.047 | 0.013 | 0.012 | 0.021 | 62.707 | −7.977 | 17.817 | −7.782 | 246.998 | 26.905 |
| | | Days | | | | | | | | | | | | |
| 5 | C | 28 | 7.886 | −4.367 | 0.06 | 0.058 | 0.033 | 0 | 54.73 | −6.684 | 10.035 | −3.905 | 273.903 | 96.089 |
| | | Days | | | | | | | | | | | | |
| 5 | C | 56 | 1.768 | | 0.105 | | 0.012 | | 56.023 | | 13.912 | | 343.087 | |
| | | Days | | | | | | | | | | | | |
| 9 | C | 0 | 3.609 | 0.978 | 0.533 | −0.328 | 0.094 | 0.026 | 60.654 | −2.085 | 2.41 | 3.784 | 413.991 | −84.132 |
| | | Days | | | | | | | | | | | | |
| 9 | C | 28 | 4.587 | −3.555 | 0.205 | −0.177 | 0.12 | 0.208 | 58.569 | −15.418 | 6.194 | 67.74 | 329.859 | 165.212 |
| | | Days | | | | | | | | | | | | |
| 9 | C | 56 | 0.054 | | 0.356 | | 0.302 | | 45.236 | | 70.15 | | 579.203 | |
| | | Days | | | | | | | | | | | | |
| 12 | C | 0 | 0.054 | 7.958 | 0.248 | 0.143 | 1.608 | 0.628 | 57.726 | 30.972 | 66.05 | 20.649 | 100.126 | 245.603 |
| | | Days | | | | | | | | | | | | |
| 12 | C | 28 | 8.012 | 0 | 0.39 | 0.048 | 2.231 | 0.806 | 88.6986 | 9.397 | 86.699 | 0 | 345.729 | 127.923 |
| | | Days | | | | | | | | | | | | |
| 12 | C | 56 | 0.054 | | 0.296 | | 2.414 | | 67.123 | | 66.05 | | 228.049 | |
| | | Days | | | | | | | | | | | | |

1. A composition comprising:
at least about 0.1% by weight cannabidiol (CBD)
at least about 40% by weight of a fatty acid component comprising a balanced ratio of an omega-3 fatty acid and an omega-6 fatty acid; and
a sterol.

2. The composition of claim 1, wherein the omega-3 acid and omega-6 fatty acid are present in a ratio of about 0.8:1 to about 1.2:1.

3. The composition of claim 2, wherein the omega-3 acid and omega-6 fatty acid are present in a ratio of about 1:1.

4. The composition of any one of claims 1 to 3, further comprising a vitamin E compound.

5. The composition of any one of claims 1 to 4, wherein the CBD has a purity of greater than 98%.

6. The composition of any one of claims 1 to 4, wherein the CBD is in the form of a Cannabis extract.

7. The composition of any one of claims 1 to 6, wherein the CBD is in an amount of up to about 10% by weight.

8. The composition of any one of claims 1 to 7, wherein the fatty acid component is present in a concentration from about 40% to about 99.9%.

9. The composition of any one of claims 1 to 8, wherein the sterol is a phytosterol.

10. The composition of claim 9, wherein the phytosterol is β-sitosterol.

11. The composition of any one of claims 1 to 10, comprising a further active pharmaceutical, veterinary and/or nutraceutical ingredient.

12. The composition of claim 11, wherein the active pharmaceutical, veterinary and/or nutraceutical ingredient is fat soluble and/or dispersible.

13. The composition of claim 11 or 12, wherein the active pharmaceutical, veterinary and/or nutraceutical ingredient is selected from an essential oil, a plant extract, a terpene, a flavonoid, bromelaine, an essential amino acid, a peptide comprising an essential amino acid, a CB2 ligand, a vitamin, oclacitinib (eg Apoquel™), a corticosteroid, a monoclonal antibody targeting a skin disorder and/or inflammation or a combination thereof.

14. The composition of any one of claims 1 to 13, further comprising β-caryophyllene.

15. The composition of any one of claims 1 to 14, comprising
cannabidiol (CBD)
a fatty acid component comprising an omega-3 fatty acid and an omega-6 fatty acid;
β-sitosterol
β-caryophyllene
a vitamin E compound; and
an antioxidant.

16. The composition of claim 15, comprising:
about 0.01% to about 10% cannabidiol (CBD)
at least about 40% of the fatty acid component comprising an omega-3 fatty acid and an omega-6 fatty acid;
about 0.01% to about 15% β-sitosterol
about 0.01% to about 1% β-caryophyllene
about 0.1% to about 10% a vitamin E compound; and
about 0.05% to about 6% an antioxidant.

17. A pharmaceutical composition comprising the composition of any one of claims 1 to 16 and a pharmaceutically acceptable diluent and/or excipient.

18. A veterinary composition comprising the composition of any one of claims 1 to 16 and a veterinary acceptable diluent and/or excipient.

19. A nutraceutical composition comprising the composition of any one of claims 1 to 16 and a nutraceutically acceptable diluent and/or excipient.

20. A method of treating a subject suffering from or at risk of developing a skin disorder and/or an inflammatory condition, comprising administering an effective amount of a composition of any one of claims 1 to 18 to a subject in need thereof.

21. The method of claim 20, comprising orally administering the composition to the subject.

22. The method of claim 20 or 21, wherein the subject is a companion animal.

23. The method of any one of claims 20 to 22, wherein the subject is a dog.
